(19) United States
(12) Patent Application Publication
(10) Pub. No.: US 2024/0221875 A1
(43) Pub. Date: Jul. 4, 2024

(54) METHODS AND APPARATUS FOR CLOUD-BASED DATA MANAGEMENT AND REPORTING FOR BIOPROCESS DEVELOPMENT

(71) Applicant: CYTIVA SWEDEN AB, Uppsala (SE)

(72) Inventors: Peter Andersson, Uppsala (SE); Alexander Kele, Uppsala (SE); Todd Ward, Marlborough, MA (US); David Henderson, Marlborough, MA (US); Vanessa Hoskins, Marlborough, MA (US); Dean Whitney, Marlborough, MA (US); Robin Modigh, Sundsvall (SE); Per LIDÉN, Uppsala (SE); Olga Szaduro, Krakow (PL); Daniel Mynarski, Krakow (PL); Adam Sipos, Uppsala (SE); Peter Cserna, Uppsala (SE); David Birkas, Uppsala (SE); Istvan Toth, Uppsala (SE); Júlia Tünde GÁL, Uppsala (SE); Hajnalka Albert, Uppsala (SE); Zsofia Vasne Utassy, Uppsala (SE); Edina Szava, Uppsala (SE); Tim Bervoets, Uppsala (SE)

(21) Appl. No.: 18/557,550

(22) PCT Filed: Apr. 29, 2022
(86) PCT No.: PCT/IB2022/054000

§ 371 Date: Oct. 26, 2023

Related U.S. Application Data
(60) Provisional application No. 63/181,776, filed on Apr. 29, 2021.

(51) Int. Cl. G16H 10/40; G16H 15/00

(52) U.S. Cl. CPC G16H 10/40; G16H 15/00

(57) ABSTRACT

Methods and apparatus for cloud-based data management and reporting for bioprocess development. The system for process data management disclosed herein includes memory and processor circuitry to execute machine readable instructions to at least retrieve a first data set from at least one of an electronic laboratory notebook or a first laboratory device, retrieve a second data set from a second laboratory device, link the first data set and the second data set to a project, the first data and the second data set linked to the project based on a type of data in the first data set and the second data set, generate a data overlay using the first data set and the second data set, the data overlay including an overlay of a first chromatogram and a second chromatogram and generate a report including the data overlay.

### RELATED APPLICATIONS

This patent arises from the national stage of International Application No. PCT/IB2022/054000, which was filed on Apr. 29, 2022, which claims priority to U.S. Provisional Patent Application Ser. No. 63/181,776, filed on Apr. 29, 2021. International Application No. PCT/IB2022/054000 and U.S. Provisional Patent Application Ser. No. 63/181,776 are hereby incorporated herein by reference in their entireties. Priority to International Application No. PCT/IB2022/054000 and Provisional Patent Application Ser. No. 63/181,776 is hereby claimed.

### FIELD OF THE DISCLOSURE

This disclosure relates generally to data management and, more particularly, to methods and apparatus for cloud-based data management and reporting for bioprocess development.

### BACKGROUND

Bioprocesses are used to produce medically and industrially critical products (e.g., therapeutics, biofuels, etc.) using biomanufacturing through optimization of natural and/or artificial biological systems to allow for large-scale production. Bioprocesses are data intensive, requiring constant documentation of ongoing procedures to ensure quality and efficiency, and support validation. Furthermore, data assessment and analysis can require the collection, integration, and visualization of data from numerous data sources across various platforms.

Bioprocesses often require real-time, continuous measurement of process variables to ensure the stability, efficiency, and reproducibility of the processes to provide for a high-quality product. By measuring quality-related process variables that are necessary to maintain a narrow range of environmental conditions, consistent reproduction of the desired product can be achieved and documented. A variety of bioprocess instruments (also referred to herein as bioprocess units) are used during upstream processing (e.g., biomass expansion, media development and preparation, etc.) and downstream processing (e.g., product extraction and purification from the biomass, etc.), including bioreactors and mixers. For example, a bioreactor can be used to create a controlled environment for in vitro management of cells (e.g., cell proliferation, differentiation, etc.) during upstream processing. Bioreactors can include sensors directly interfacing, or used in conjunction with, the bioreactors to measure process variables, including oxygen and carbon dioxide concentration, biomass concentration, flow injection, and/or overall media composition. Downstream processes focus on optimizations to extract and maximize final product yields, including filtration, mixing, and purification based on chromatography.

Bioprocesses (e.g., use of living cells and/or cell components to obtain products such as biotherapeutics) can be developed at smaller scales before stepwise transfer to larger volumes occurs to achieve industrial production-scale levels (e.g., scaling up based on bioreactor operating parameters from a smaller scale to a larger scale the process is transferred to). Reliable bioprocess scaling up is needed to achieve consistent products of high quality, including high product yields. During a given bioprocess, a large amount of data is stored, communicated, and shared across various platforms that are not integrated. For example, multiple individuals (e.g., analytical scientist(s), process development scientist(s), technical project leader(s), etc.) may need to access, modify, and/or validate data collected over the duration of a given bioprocess or afterwards. Every individual can use multiple methods of data collection, assessment, and/or storage (e.g., Excel file, electronic laboratory notebook (ELN), etc.). However, there is a need for a centralized data management system that would provide a single platform for collecting, integrating, and/or evaluating data relevant to a given process that can also be user-specific (e.g., based on the data assessment and/or data evaluating needs of an analytical scientist versus a process development scientist, etc.).

### BRIEF SUMMARY

Certain examples provide methods and apparatus for cloud-based data management and reporting for bioprocess development. Certain examples provide a system for process data management, the system including memory and processor circuitry to execute machine readable instructions to at least retrieve a first data set from an electronic laboratory notebook or a first laboratory device and retrieve a second data set from a second laboratory device. The example system also includes processor circuitry to execute machine readable instructions to at least link the first data set and the second data set to a project, the first data and the second data set linked to the project based on a type of data in the first data set and the second data set and generate a data overlay using the first data set and the second data set, the data overlay including an overlay of a first chromatogram and a second chromatogram. The example system also includes processor circuitry to execute machine readable instructions to at least generate a report including the data overlay, the data overlay a result of a bioprocessing step associated with the project, link the report to the project, the project accessible to at least two users associated with the project, and track changes made by the at least two users to the first data set or the second data set associated with the report.

Certain examples provide a method for process data management, the method including retrieving a first data set from an electronic laboratory notebook or a first laboratory device and retrieving a second data set from a second laboratory device. The example method also includes linking the first data set and the second data set to a project, the first data and the second data set linked to the project based on a type of data in the first data set and the second data set, generating a data overlay using the first data set and the second data set, the data overlay including an overlay of a first chromatogram and a second chromatogram, and generating a report including the data overlay, the data overlay a result of a bioprocessing step associated with the project. The example method also includes linking the report to the project, the project accessible to at least two users associated with the project and tracking changes made by the at least two users to the first data set or the second data set associated with the report.

Certain examples provide at least one computer readable storage medium including instructions that, when executed, cause at least one processor to retrieve a first data set from an electronic laboratory notebook or a first laboratory device and retrieve a second data set from a second laboratory device. The example instructions further cause the at least one processor to link the first data set and the second data set to a project, the first data and the second data set linked to the project based on a type of data in the first data set and the second data set, generate a data overlay using the first data set and the second data set, the data overlay including an overlay of a first chromatogram and a second chromatogram, and generate a report including the data overlay, the data overlay a result of a bioprocessing step associated with the project. The example instructions further cause the at least one processor to link the report to the project, the project accessible to at least two users associated with the project and track changes made by the at least two users to the first data set or the second data set associated with the report.

### BRIEF DESCRIPTION OF THE DRAWINGS

FIG. 1 illustrates example compilation and analysis of bioprocess data as the data is collected, analyzed, and updated over time by various members of a project team.

FIG. 2 illustrates an example interaction among several systems used for collecting and analyzing bioprocess data, including electronic laboratory notebook(s) and device(s) used to obtain bioprocess data, as well as the management of data from these sources by a process development central data management system.

FIG. 3 is a block diagram illustrating an example process development central data management system in accordance with the teachings of this disclosure.

FIG. 4 is a flowchart representative of example machine-readable instructions that may be executed by the process development central data management system.

FIG. 5 illustrates an example collection of data for integral data management.

FIG. 6 illustrates an example application screen overview used for creating and editing projects based on the process development central data management system of FIG. 3.

FIG. 7A illustrates an example application screen overview used for viewing and/or managing preparative data and/or creating overlays of existing data associated with the process development central data management system of FIG. 3.

FIG. 7B illustrates an example application screen overview used for viewing and/or managing preparative data and/or re-arranging preparative chromatograms associated with the process development central data management system of FIG. 3.

FIG. 8A illustrates an example application screen overview used for data pairing associated with the process development central data management system of FIG. 3.

FIG. 8B illustrates an example application screen overview used for chromatogram overlays performed in connection with the process development central data management system of FIG. 3.

FIG. 9 illustrates an example application screen overview used for managing process data chromatograms in connection with the process development central data management system of FIG. 3.

FIG. 10 illustrates an example application screen overview used for downloading process data chromatograms in connection with the process development central data management system of FIG. 3.

FIG. 11 illustrates an example architecture implemented in connection with the process development central data management system of FIG. 3.

FIG. 12 illustrates an example architecture used for logging and monitoring events related to deployment, infrastructure, applications and security in connection with the process development central data management system of FIG. 3.

FIG. 13 illustrates an example architecture used for uploading files and data processing in connection with the process development central data management system of FIG. 3.

FIG. 14 illustrates an example architecture used for accessing data in connection with the process development central data management system of FIG. 3.

FIG. 15 is a block diagram of an example processing platform structured to execute the example instructions of FIG. 4 to implement the example process development central data management system of FIGS. 2-3.

FIG. 16 is a block diagram of an example implementation of the processor circuitry of FIG. 15.

FIG. 17 is a block diagram of another example implementation of the processor circuitry of FIG. 15.

FIG. 18 is a block diagram of an example software distribution platform (e.g., one or more servers) to distribute software (e.g., software corresponding to the example machine readable instructions of FIG. 4) to client devices associated with end users and/or consumers (e.g., for license, sale, and/or use), retailers (e.g., for sale, re-sale, license, and/or sub-license), and/or original equipment manufacturers (OEMs) (e.g., for inclusion in products to be distributed to, for example, retailers and/or to other end users such as direct buy customers).

The figures are not scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

Methods and apparatus for cloud-based management of bioprocesses, such as chromatography, described herein permit the implementation of a secure, cloud-based management scheme that represents a single platform with a centralized database for collecting, integrating, and visualizing data relevant to a given process and/or user. Methods and apparatus disclosed herein permit integrated data analysis and assessment, streamlined data reporting, and a platform-wide, accessible database of stored results. As such, methods and apparatus disclosed herein reduce the time for data processing and/or analysis, increase data integrity and traceability, and secure data for re-use across an entire organization. In the examples disclosed herein, a process development central software tool can collect data from different process (e.g., science and analytical) lab devices in upstream and downstream process development.

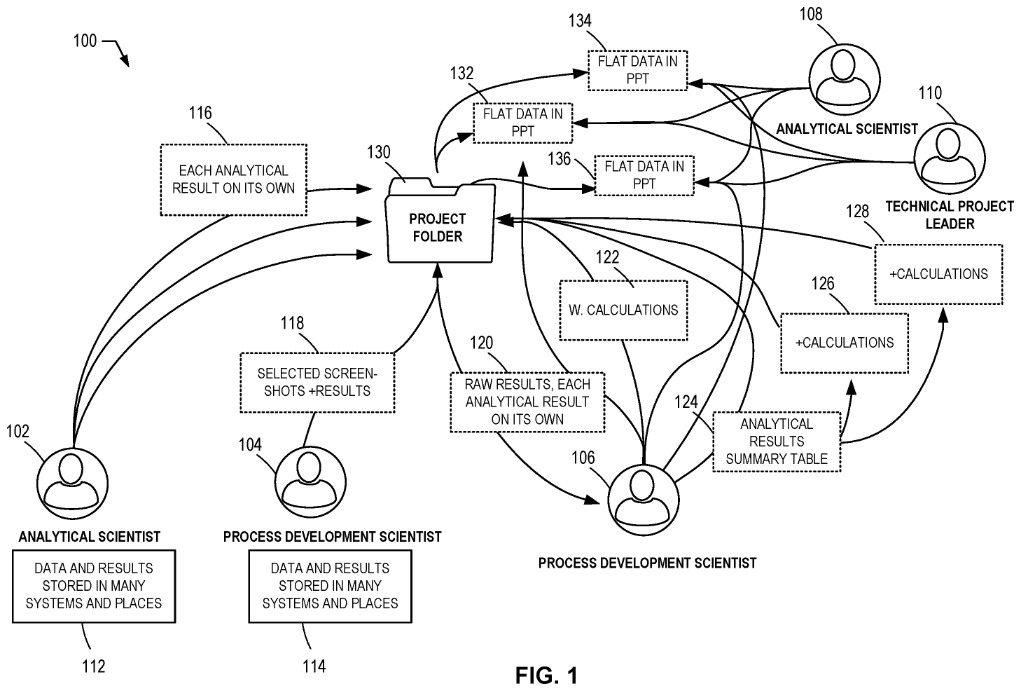

FIG. 1 illustrates example data flow 100 including compilation and analysis of bioprocess data as the data is collected, analyzed, and updated over time by various members of a project team. Known systems for process-based data management can include platforms from many vendors and with different data formats. For example, multiple individuals may be working on and/or contributing to a given process (e.g., data interpretation, data assessment, monitoring, raw result gathering, data report sharing, etc.). As shown in the example of FIG. 1, interactions can occur among analytical scientists (e.g., example analytical scientist(s) 102, 108), process development scientists (e.g., example process development scientist(s) 104, 106), and/or technical project leader(s) (e.g., example technical project leader 110). Scientists can use many different methods and/or instruments from different manufacturers in parallel. In the prior art, data from different platforms is integrated manually and/or manually contextualized for joint analysis (e.g., using Excel). As such, data management issues can arise as the data gathering is time-consuming and can be difficult to find, retrieve, and/or understand (e.g., given that the data is not stored in a standardized format and/or in a flat format).

In the example of FIG. 1, the analytical scientist 102 and/or the process development scientist 104 can have data and/or results stored in many different systems and/or places (e.g., example data 112, 114). Furthermore, although the scientist(s) 102, 104, 106, 108, and/or 110 can be working on the same project (e.g., example project folder 130), each individual contributing to the assessment and retrieval of data can have data capture using different platforms and/or applications, with each analytical result independent of the other results (e.g., including calculations) provided to the project folder 130 and/or shared with other collaborators (e.g., example data 116, 118, 120, 122, 124). In some examples, the data is updated and/or modified over time as more individuals review it (e.g., adding example calculations 126, 126). Likewise, the data can be retrieved from the project folder 130 in different formats (e.g., example flat data in a PowerPoint format 132, 134, 136). As such, it can be difficult to report and/or share data as needed. For example, it can be time consuming for a collaborator to compile various data reports where results are maintained in a flat format (e.g., a Word and/or PowerPoint document). For example, in such flat formats, too little context may be presented such that the collaborator and/or supervisor (e.g., technical project leader 110) is not able to evaluate the raw data directly.

The impracticalities associated with such known methods of data management as presented in FIG. 1 result in low efficiency and increased cost. For example, a lack of cross-functional collaboration and increased time spent on manual data shifting, building home-grown tools, and maintaining old software contribute to the decreased efficiency and cost increases. Likewise, reliance on individuals and/or tribal knowledge contributes to high losses during handovers (e.g., transferring of data from one group to another), thereby increasing risk and decreasing overall quality of the data management. For example, manual handovers of data can make it cumbersome to keep context and/or maintain data integrity. Over time, competitive innovation can be reduced due to loss of, and/or a lack of access to, historical data. Lack of historical information hinders the implementation of artificial intelligence-based technologies (e.g., applications of artificial intelligence for data management purposes, etc.) and/or implementation of cloud-based technologies.

Methods and apparatus disclosed herein permit the collection of all relevant data for a combined assessment and/or analysis of the data for upstream and/or downstream processes. As such, the methods and apparatus disclosed herein permit a reduction in time spent on error prone manual data integration, report creation, preparation and/or editing of reports to make data presentable, and/or overall data preparation for analysis. Additionally, methods and apparatus disclosed herein permit data to be secured through the storage of data within a relevant context in a single location that allows for increased data integrity so that it can be easily identified, accessed, and/or re-used across a single organization.

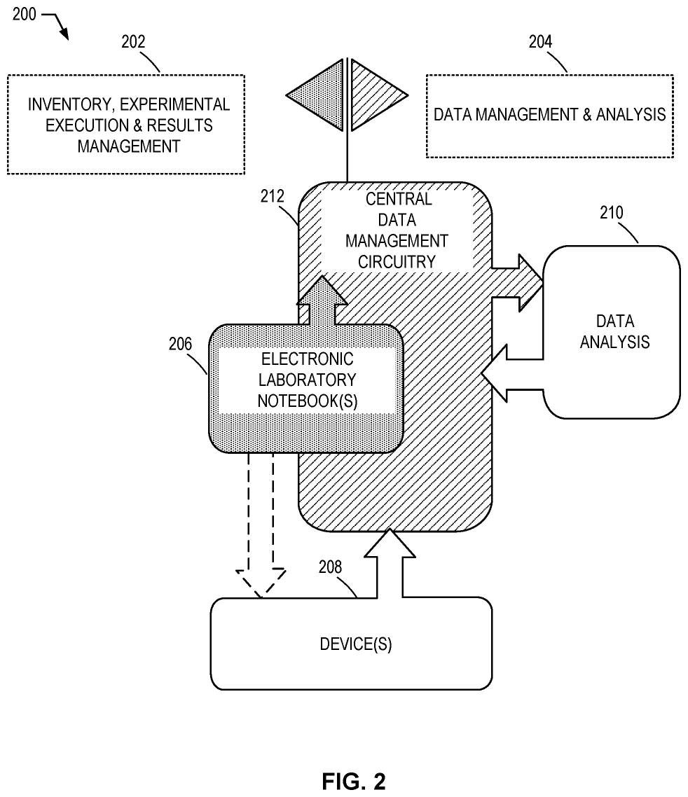

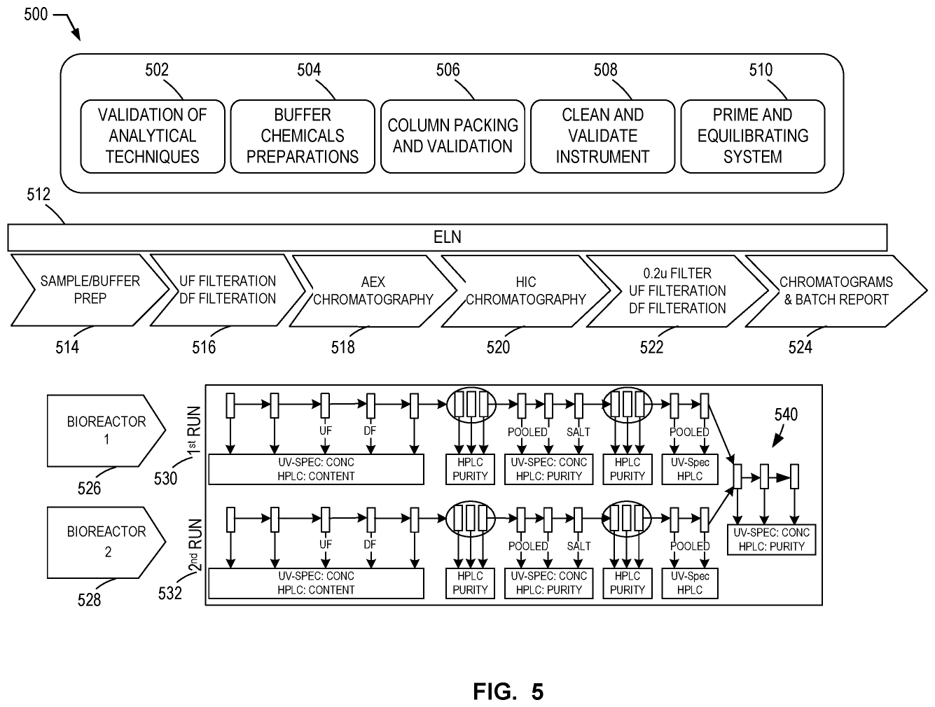

FIG. 2 illustrates an example interaction 200 among several systems used for collecting and analyzing bioprocess data, including example electronic laboratory notebook(s) 206 and example device(s) 208 used to obtain bioprocess data, as well as the management of data from these sources by an example process development (PD) central data management circuitry 212. In the example of FIG. 2, the PD central data management circuitry 212 (also interchangeably referred to herein as data management circuitry 212) can receive input from the electronic laboratory notebook(s) 206 and/or example device(s) 208 (e.g., instruments used to generate data, etc.). For example, the PD central data management circuitry 212 can obtain example inventory, experimental execution, and/or results management information 202 (e.g., provided by the ELNs 206), while allowing for overall data management and analysis 204. As shown in the example of FIG. 2, the process development (PD) central data management circuitry 212 can contribute information, for example, data analysis 210, while also receiving information from any generated data analysis 210 that can be performed externally to the process development (PD) central data management circuitry 212. The organization of the data flow as shown in the example of FIG. 2 permits for a more centralized data management system that allows for direct retrieval of data from existing sources (e.g., ELNs 206) while providing additional tools for the management and analysis of the retrieved data. As shown in connection with FIG. 5, the PD central data management circuitry 212 can assist in the processing of data associated with a bioprocess (e.g., using bioreactor(s), which can represent device(s) 208 of FIG. 2). For example, data can originate from ultraviolet spectrophotometer (UV-Spec) and/or high-performance liquid chromatography (HPLC). In some examples, the ELNs 206 can include data related to sample/buffer preparation, filtration, chromatography, and/or batch reports. The (PD) central data management circuitry 212 permits the combination of data collection and analysis related to analytical testing (e.g., bioassays, spectrophotometry, chromatography, etc.) and the upstream and/or downstream process platforms such as UNICORN® (e.g., control system for real-time control of protein purification unit operations including column packing, chromatography, and/or filtration). In some examples, the PD central data management circuitry 212 can be used to contextualize, integrate, and/or visualize data (e.g., such as data received from UNICORN®, etc.). Using the PD central data management circuitry 212, reports can be generated, exported, and/or shared with collaborators. Additionally, and as illustrated by the disclosure, this further allows for optimization of bioprocessing steps (e.g., by contextualizing, integrating, and/or visualizing the data modifications to optimize the process are more readily identified and implemented).

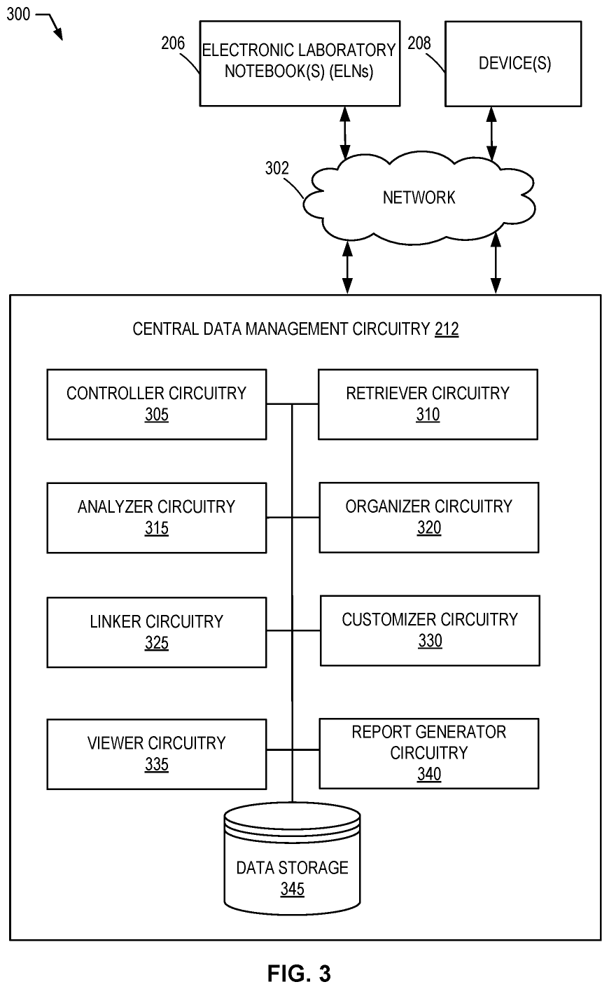

FIG. 3 is a block diagram 300 illustrating the process development central data management circuitry 212 in accordance with the teachings of this disclosure. In the example of FIG. 3, the PD central data management circuitry 212 of FIG. 2 receives input (e.g., via an example network 302) of data from the electronic laboratory notebook(s) 206 of FIG. 2 and/or the device(s) 208 (e.g., bioreactors, etc.). In the example of FIG. 3, the PD central data management circuitry 212 includes an example controller circuitry 305, an example retriever circuitry 310, an example analyzer circuitry 315, an example organizer circuitry 320, an example linker circuitry 325, an example customizer circuitry 330, an example viewer circuitry 335, an example report generator circuitry 340, and/or an example data storage 345.

The controller circuitry 305 controls the data management process of the PD central data management circuitry 212. For example, the controller circuitry 305 identifies the source of data input (e.g., ELNs 206, device(s) 208) and/or type of data input (e.g., file type, size, etc.). In some examples, the data input received from the electronic laboratory notebook(s) (ELNs) 206 and/or the device(s) 208 is maintained in the native file structure without the need for parsing and/or standardization. For example, instead of processing the data to input the received data into a relational database, the data can be maintained as is (e.g., in a native format), while additional code can be added to be able to process and/or handle the data using the PD central data management circuitry 212. In some examples, the controller circuitry 305 determines when data needs to be retrieved (e.g., using the retriever circuitry 310), when data should be analyzed based on user input(s) and/or selections (e.g., using the analyzer circuitry 315), and/or determines whether the organizer circuitry 320, the linker circuitry 325, the customizer circuitry 330, the viewer circuitry 335 and/or the report generator circuitry 340 should be engaged based on the user-provided input and/or selection. In some examples, the controller circuitry 305 determines a sequence of steps to be performed by the PD central data management circuitry 212 based on the data input(s) (e.g., from the ELNs 206 and/or the device(s) 208). In some examples, the controller circuitry 305 can be used to update software-specific features related to the PD central data management circuitry 212.

In some examples, the controller circuitry 305 initializes a connection with the electronic laboratory notebook(s) (ELNs) 206 and/or the device(s) 208 (e.g., via a communication interface). For example, the controller circuitry 305 can initialize a connection with a local device (e.g., a device located in proximity to and/or connected to a same network as the controller circuitry 305, etc.). In some examples, the controller circuitry 305 permits the PD central data management circuitry 212 to connect to the electronic laboratory notebook(s) (ELNs) 206 and/or the device(s) 208 using a transmission control protocol (TCP) handshake and/or automatic handshaking to allow for the exchange of data between the data management circuitry 212 (e.g., via the controller circuitry 305) and the electronic laboratory notebook(s) (ELNs) 206 and/or the device(s) 208. In some examples, if the controller circuitry 305 initiates a request to connect to a local device, the controller circuitry 305 can initiate a CONNECT request to establish a connection with a remote endpoint. In some examples, the local device can confirm the connection and/or the controller circuitry 305 can send an additional request to confirm the status of the local device (e.g., device(s) 208). As such, any information provided by the electronic laboratory notebook(s) (ELNs) 206 and/or the device(s) 208 can be received by the PD central data management circuitry 212 via the controller circuitry 305.

In some examples, the controller circuitry 305 can also be used to provide a data source system (e.g., electronic laboratory notebook(s) (ELNs) 206 and/or the device(s) 208) with data, instructions, and/or updates. For example, any reports generated (e.g., using the report generator circuitry 340) can be provided to the electronic laboratory notebook(s) (ELNs) 206 and/or the device(s) 208 via the controller circuitry 305. As described in the methods and apparatus disclosed herein, the information provided to the electronic laboratory notebook(s) (ELNs) 206 and/or the device(s) 208 can include chromatogram overlays, visualizations of results, data analytics, and/or integration of multiple data files (e.g., data analytics) in parallel. In some examples, the PD central data management circuitry 212 can connect (e.g., via an automatic handshake) to the data source system and can further create task(s) relevant to a given data transfer request, determine applicable parameter(s), and/or iterate to adjust parameters as required by the equipment and/or systems at both ends (e.g., the data management system versus the data source system). For example, handshaking can permit the data management circuitry 212 and the data source system to negotiate parameters such as information transfer rate, interrupt procedure(s), and/or other protocol features. In some examples, methods and apparatus disclosed herein rely on an application programming interface (API) such as a representational state transfer (REST) API (e.g., that conforms to the constraints of a REST architecture and/or allows for application-based interaction with RESTful web services via TCP/IP). For example, the PD central data management circuitry 212 can connect to a remote system using a documented API and/or using a third-party broker (e.g., a connectivity partner).

The retriever circuitry 310 retrieves data from the data sources available to the PD central data management circuitry 212. For example, the retriever circuitry 310 can retrieve data from the ELNs 206 and/or the device(s) 208. In some examples, the retrieving of the data is based on user-initiated uploads of files exported from ELNs 206 or files created by a user based on data. In some examples, the retriever circuitry 310 retrieves data based on a user request for specific information and/or based on a request to import data specific to a given project. In some examples, the retriever circuitry 310 retrieves data specific to a given instrument (e.g., chromatography, spectrophotometry, etc.). In some examples, the retriever circuitry 310 retrieves data based on a specified time interval and/or a specific collaborator who performed a given data analysis and/or collected a given set of data of interest to a user. In some examples, the retriever circuitry 310 retrieves and stores (e.g., using the data storage 345) data from any available source in communication with the PD central data management circuitry 212 (e.g., via the network 302). In the example of FIG. 3, the network 302 can be implemented using any suitable wired and/or wireless network(s) including, for example, one or more data buses, one or more Local Area Networks (LANs), one or more wireless LANs, one or more cellular networks, the Internet, etc. As used herein, the phrase “in communication,” including variances thereof, encompasses direct communication and/or indirect communication through one or more intermediary components and does not require direct physical (e.g., wired) communication and/or constant communication, but rather additionally includes selective communication at periodic or aperiodic intervals, as well as one-time events.

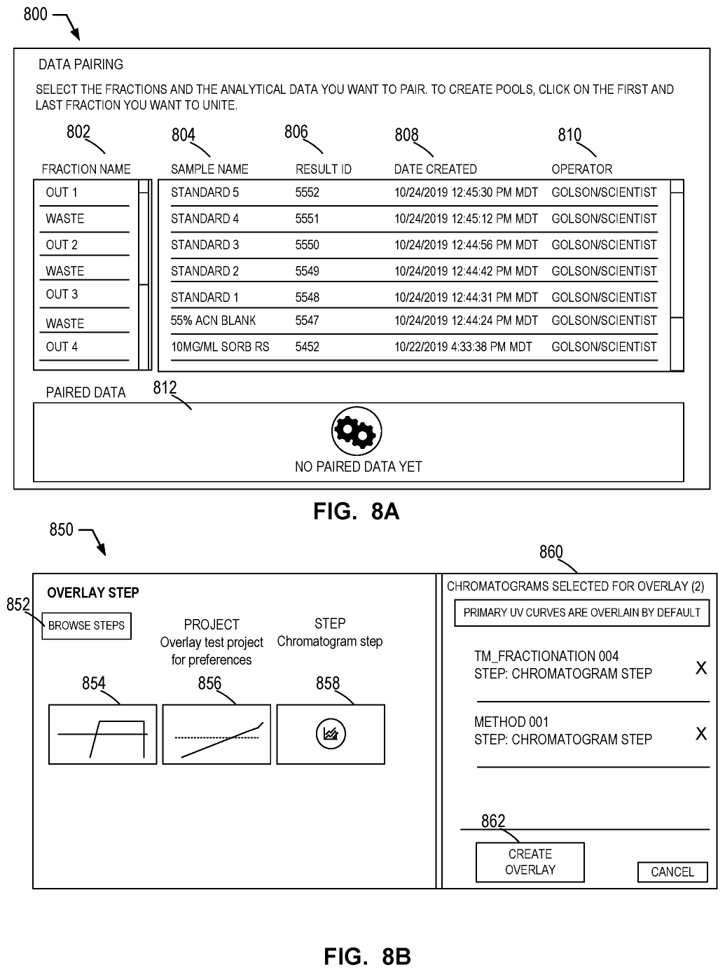

The analyzer circuitry 315 performs data analysis based on data retrieved by the PD central data management circuitry 212 using the retriever circuitry 310. In some examples, the analyzer circuitry 310 performs data analysis based on user-defined preferences (e.g., statistical assessments, assessments of chromatograms, etc.). For example, the analyzer circuitry 315 can be used to overlay chromatographs, as described in connection with FIG. 8B. In some examples, the analyzer circuitry 315 can perform any type of data assessment needed based on a given data set. For example, the analyzer circuitry 315 can perform assessments of imported data (e.g., retrieved from the ELNs 206) and/or overlayed chromatograms.

The organizer circuitry 320 organizes data in the PD central data management circuitry 212 and/or allows a user to modify data based on desired assessments and/or final outputs (e.g., type of data output, such as graphical and/or tabulated, etc.). In some examples, the organizer circuitry 320 permits organization of a specific data type (e.g., changes in x- and/or y-axes on a graph, changes to titles, etc.). As shown in connection with FIG. 8A, the organizer circuitry 320 can be used to create a chromatography step, create an overlay step, manage process data and/or overlays, reorder chromatograms, include descriptions, edit descriptions, include comments, and/or import supplementary files into a project step. Additionally, the organizer circuitry 320 can be used to pair analytical data with process data chromatogram fractions and/or fraction pools in a chromatography project step.

The linker circuitry 325 links available data accessible via the PD central data management circuitry 212 and makes the data searchable and ready for analysis. For example, by allowing for the data to be searchable, the linker circuitry 325 assists in the integration of data across the entire PD central data management circuitry 212. As such, a user can simply search for a specific data entry and/or data linked to a particular project based on the data linking performed via the linker circuitry 325. In some examples, the linker circuitry 325 can link specific data types (e.g., graphs, etc.) based on what type of data a user is searching for and/or attempting to view. In some examples, the linker circuitry 325 can be used to identify data sets that belong to a specific project for improved data sharing. In some examples, the linker circuitry 325 can perform a priori linking of one data set to another (e.g., analytical results paired to a fraction). In some examples, the linker circuitry 325 can link data as part of a search (e.g., perform linking and/or make associations in real time based on inferences).

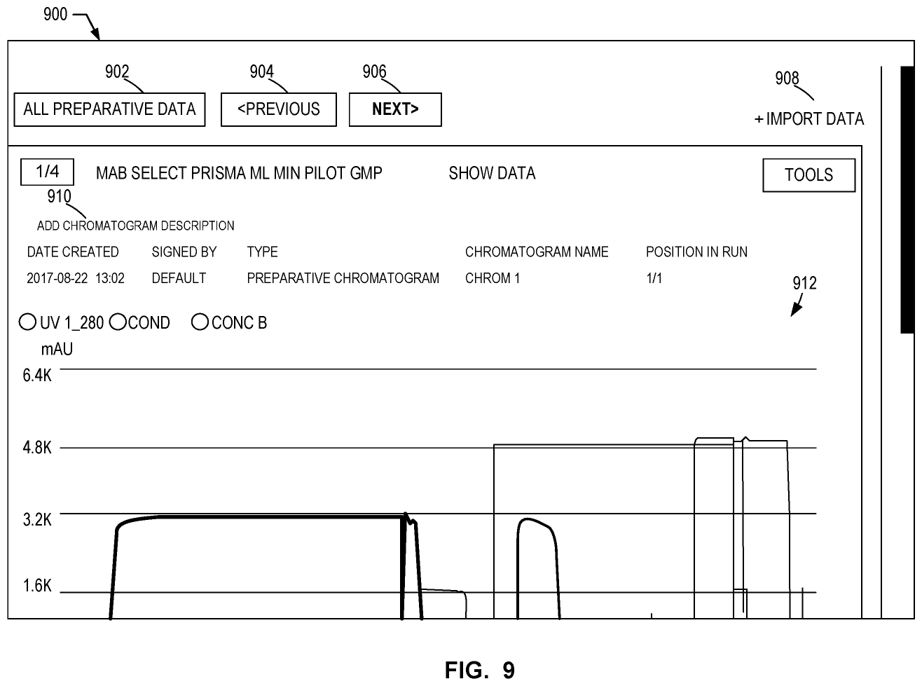

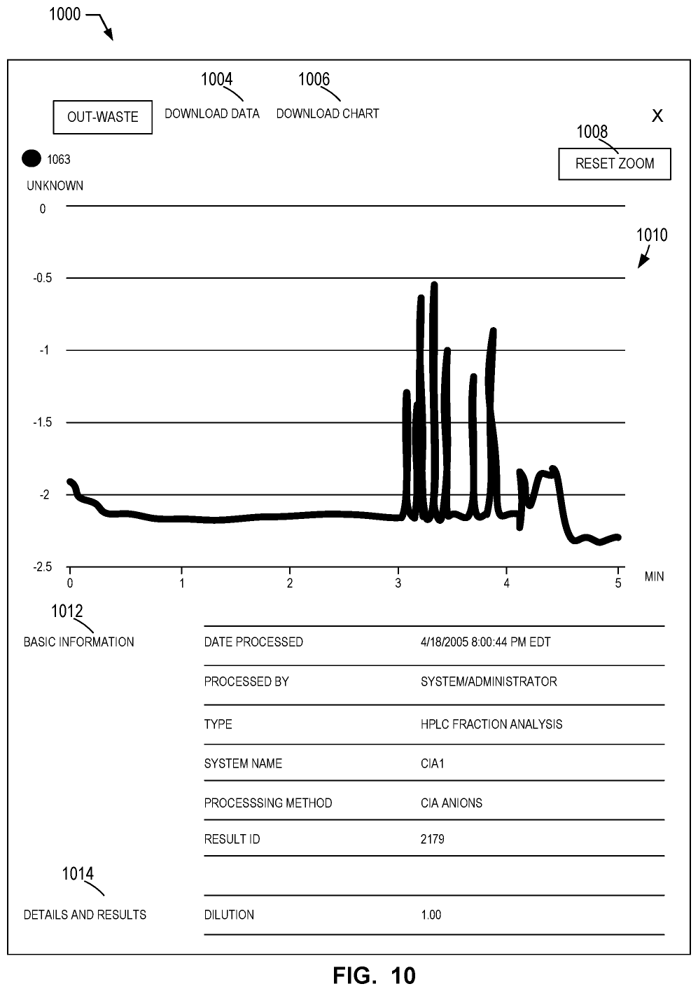

The customizer circuitry 330 customizes various aspects of the user interaction with the PD central data management circuitry 212. In some examples, the customizer 330 can be used to create and edit projects. In some examples, the customizer circuitry 330 can be used to manage chromatograms by allowing modifications of the chromatograms based on user preferences, as described in connection with FIG. 9 and/or FIG. 10. In some examples, the customizer circuitry 330 can be used to manage and/or organize a user profile.

The viewer circuitry 335 provides a user of the PD central data management circuitry 212 with views of selected data in various forms (e.g., graphical, tabulated, etc.). In some examples, the viewer circuitry 335 provides previews of generated data prior to exporting the data to the ELNs 206. In some examples, the viewer circuitry 335 provides the user with views of chromatogram overlays. In some examples, the viewer circuitry 335 can be used to view analytical data that is imported into a given chromatogram. Furthermore, the viewer circuitry 335 can display process data chromatogram data important into a selected step, as shown in connection with FIG. 9. For example, the viewer circuitry 335 can be used to view chromatograms when customizing curves (e.g., using the customizer circuitry 330), selecting which curves are displayed in the chart, changing the x- and/or y-axis base unit (e.g., to a time or to a volume), zooming within the graph, and/or highlighting one or more curve(s).

The report generator circuitry 340 generates reports within the PD central data management circuitry 212. In some examples, reports generated by the report generator circuitry 340 can be exported to the ELNs 206. For example, the report generator circuitry 340 can be used to include information such as overlay of one data set and/or graph over another data set and/or graph (chromatogram overlay) to observe convergences and/or divergences of data among different experiments and/or data sets. In some examples, the report generator circuitry 340 can be used to create a project report in any type of format (e.g., including DOCX, PPTX, etc.). In some examples, the report generator circuitry 340 generates a report that includes project information, project steps, all imported data (e.g., process/analytical chromatogram charts, metadata, etc.), and/or any pairing information.

The data storage 345 stores any data associated with the controller circuitry 305, the retriever circuitry 310, the analyzer circuitry 315, the organizer circuitry 320, the linker circuitry 315, the customizer circuitry 330, the viewer circuitry 335, and/or the report generator circuitry 340. The data storage 345 may be implemented by any storage device and/or storage disc for storing data such as, for example, flash memory, magnetic media, optical media, etc. Furthermore, the data stored in the data storage 345 may be in any data format such as, for example, binary data, comma delimited data, tab delimited data, structured query language (SQL) structures, etc. While in the illustrated example the data storage 345 is illustrated as a single database, the data storage 345 can be implemented by any number and/or type(s) of databases.

While an example implementation of the process development central data management circuitry 212 is illustrated in FIG. 3, one or more of the elements, processes and/or devices illustrated in FIG. 3 may be combined, divided, re-arranged, omitted, eliminated and/or implemented in any other way. Further, the example controller circuitry 305, the example retriever circuitry 310, the example analyzer circuitry 315, the example organizer circuitry 320, the example linker circuitry 325, the example customizer circuitry 330, the example viewer circuitry 335, the example report generator circuitry 340, and/or, more generally, the example process development central data management circuitry 212 of FIG. 3 may be implemented by hardware, software, firmware and/or any combination of hardware, software and/or firmware. Thus, for example, any of the example controller circuitry 305, the example retriever circuitry 310, the example analyzer circuitry 315, the example organizer circuitry 320, the example linker circuitry 325, the example customizer circuitry 330, the example viewer circuitry 335, the example report generator circuitry 340, and/or, more generally, the example process development central data management circuitry 212 can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example controller circuitry 305, the example retriever circuitry 310, the example analyzer circuitry 315, the example organizer circuitry 320, the example linker circuitry 325, the example customizer circuitry 330, the example viewer circuitry 335, the example report generator circuitry 340, and/or, more generally, the example process development central data management circuitry 212 of FIG. 3 is/are hereby expressly defined to include a non-transitory computer readable storage device or storage disk such as a memory, a digital versatile disk (DVD), a compact disk (CD), a Blu-ray disk, etc. including the software and/or firmware. Further still, the example process development central data management system of FIG. 3 may include one or more elements, processes and/or devices in addition to, or instead of, those illustrated in FIG. 3, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

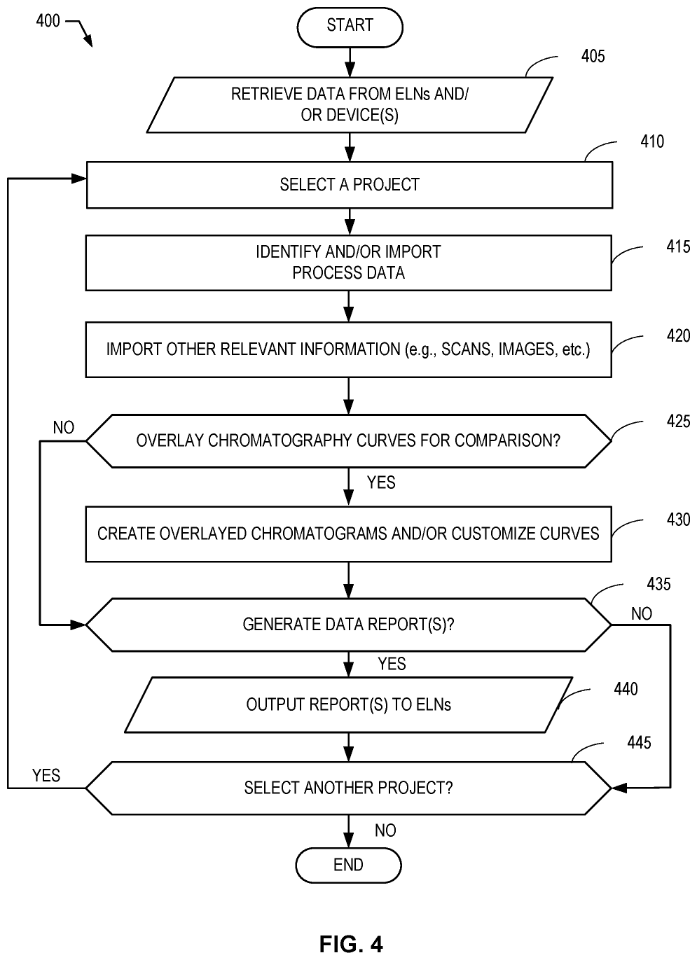

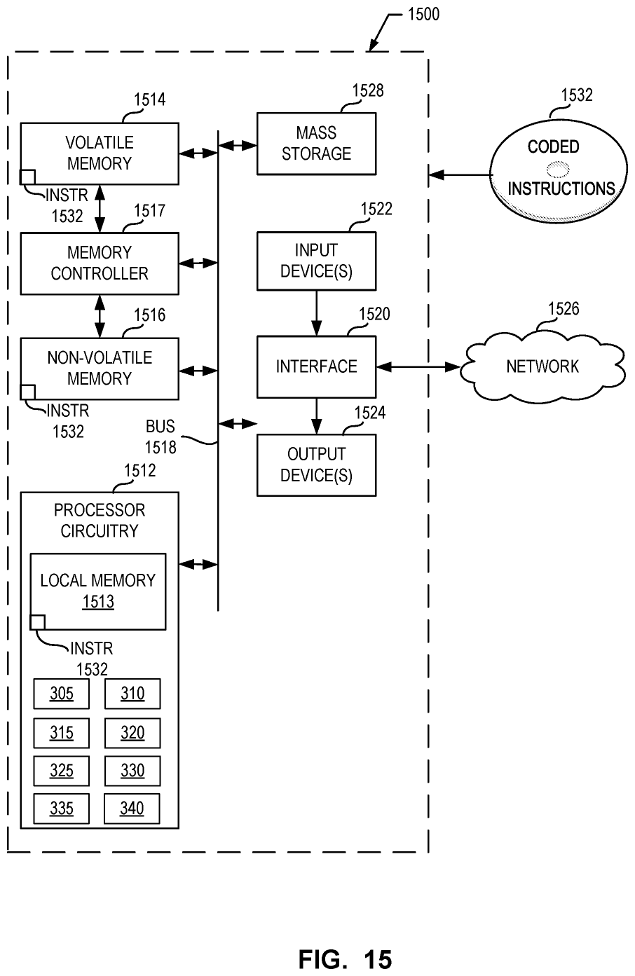

A flowchart representative of example hardware logic, machine readable instructions, hardware implemented state machines, and/or any combination thereof for implementing the process development central data management circuitry 212 of FIG. 3 is shown in FIG. 4. The machine readable instructions may be one or more executable programs or portion(s) of an executable program for execution by a computer processor such as the processor 1512 shown in the example processor platform 1500 discussed below in connection with FIG. 15. The program may be embodied in software stored on a non-transitory computer readable storage medium such as a CD-ROM, a floppy disk, a hard drive, a DVD, a Blu-ray disk, or a memory associated with the processor 1512, but the entire program and/or parts thereof could alternatively be executed by a device other than the processor 1512 and/or embodied in firmware or dedicated hardware. Further, although the example program is described with reference to the flowchart illustrated in FIG. 4, many other methods of implementing the example process development central data management circuitry 212 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C #, Perl, Python, JavaScript, HyperText Markup Language (HTML), Structured Query Language (SQL), Swift, Ladder Logic, Function Block Diagram (FBD), Structured Text, Sequential Flow Charts, Instruction List, etc.

As mentioned above, the example process of FIG. 4 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

FIG. 4 is a flowchart representative of example machine-readable instructions 400 that may be executed by the process development central data management circuitry 212. In the example of FIG. 4, the retriever circuitry 310 retrieves data from the electronic laboratory notebooks (e.g., bioreactors, etc.) in communication with the PD central data management circuitry 212 (e.g., via the network 302) (block 405). In some examples, the retrieved data can include raw data from the device(s) 208. In some examples, the retrieved data can include data (e.g., calculations, experimental results, analytical results, etc.) stored in an ELN 206 by an analytical scientist 102, a process development scientist 104, and/or a technical project leader 110 of FIG. 1. If the data is retrieved from a local device and/or another data source system (e.g., ELNs 206 and/or device(s) 208), the controller circuitry 305 of the data management circuitry 212 can initiate contact with the data source system (e.g., via a CONNECT request, etc.). For example, a communication interface of the data management circuitry 212 can connect to the device(s) 208 using an automatic handshake. During the handshake, tasks and/or associated parameter(s) can be generated and refined between the data management circuitry 212 and the data source system. In some examples, the handshaking and/or data exchange can occur between the data management circuitry 212 and several data source systems at once (e.g., multiple devices communicating with the data management circuitry 212 in parallel). However, retrieval of data is not limited to ELNs 206 and/or device(s) 208 of FIG. 2, and communication to retrieve data can be initiated with any other data source(s) and/or applications for managing and/or interacting with data (e.g., software such as UNICORN® and/or Empower). For example, the data management circuitry 212 can be based on the use of data objects (e.g., UNICORN® runs and/or spreadsheets), data uses (e.g., references) that link data objects to projects and/or steps, and/or data sources (e.g., ELNs 206, device(s) 208). For example, data sources (e.g., ELNs 206) can be integrated at a tenant level and/or linked to specific projects and/or studies (e.g., to narrow the scope). In some examples, the data management circuitry 212 described herein enables users to link the data objects (e.g., UNICORN® runs and/or spreadsheets) to projects, studies, and/or steps as well as to each other (e.g., linking of data objects). Such linking permits users to create contextualized data needed for analytics and/or reporting. For example, data objects can be initially imported into a holding and/or staging area and then linked. In other examples, the data objects can be imported directly into a project step. Additionally, the data linkage can be performed manually and/or via an orchestration that is aware of the project steps and thereby automatically links incoming data to the correct section of a given project. In some examples, the data management circuitry 212 includes search tools and/or network diagrams to indicate how data elements and/or project steps relate to each other. The data can be copied directly into the data management circuitry 212 after retrieval and/or the data can be maintained in its original location and simply linked (e.g., using a federated data model, etc.). In some examples, data sources (ELNs 206, device(s) 208) can be configured to push data into the data management circuitry 212. In some examples, the data management circuitry 212 can be linked to data sources and pull data from the data source(s) (e.g., ELNs 206, device(s) 208).

Once a connection between the data source system and the data management circuitry 212 has been established, the data management circuitry 212 can permit a user to select a project (block 410). In some examples, data retrieval from the ELNs 206 and/or devices 208 is not necessary prior to the selection of a project. For example, the user can directly access a given project before data is available or has been imported (e.g., data can become available in real-time as the user is accessing the project and/or at a later time). Also, a user can create and or access a given project prior to data retrieval so that process steps or tasks can be set up that correspond to specific process data. As such, the data management circuitry 212 set-up can be completed before project-based data is available, thereby providing a streamlined mechanism to organize and retrieve specific data. In some examples, the viewer circuitry 335 can be used to view existing projects available via the PD central data management circuitry 212 (e.g., stored in the data storage 345 and/or accessible via the network 302). In some examples, the user can use the retriever circuitry 310 to identify and/or import specific data of interest (e.g., chromatography data, other process data) (block 415). For example, the retriever circuitry 310 can retrieve (e.g., view and/or import) chromatography-based data from the ELNs 206 and/or devices 208, if the chromatography data is not accessible via the data storage 345. In some examples, the retriever circuitry 310 can be used to import process data specific to a project of interest (block 420). In some examples, other relevant information can be imported (e.g., scans, images, etc.). For example, additional imported data can include gel scan images, high performance liquid chromatography (HPLC) images, etc.

In some examples, the analyzer circuitry 315 can be used to obtain analytical data based on available results and/or experimental values. Additionally, a user can select to overlay chromatography curves for comparison (block 425). For example, the user may need to determine any divergences and/or convergences among several chromatograms. The analyzer circuitry 315 can be used to compare the curves and/or create overlays, while the customizer circuitry 330 can be used to modify x- and/or y-axes associated with the curves and/or select specific regions of the curves for comparison (block 430). This capability provides an easy and convenient mechanism to visualize and compare multiple curves without manual scaling of the data. Once any necessary assessments are performed, the user can determine whether to generate a data report (block 435). The report generator circuitry 340 can be used to generate reports and/or export the reports to the ELNs 206 (block 440). The user can determine whether to select another project to work on (block 445), at which point control returns to block 410. Furthermore, the user can access the viewer circuitry 335 to visualize any of the generated reports and/or any results obtained as a result of associating different data sets. In some examples, the viewer circuitry 335 can also be used to turn on and/or turn off different curves for viewing based on specified preferences. As such, data can be integrated using the PD central data management circuitry 212, allowing various users (e.g., analytical scientists, project managers, etc.) to access the system and retrieve data in a consistent format while ensuring secure data sharing and processing.

While an example execution of the process development central data management circuitry 212 is shown in FIG. 4, the process development central data management circuitry 212 can be used to execute any steps associated with the organization and/or management of process data. In some examples, the PD central data management circuitry 212 can proceed with linking existing data and/or preparing the user interface for data importing prior to the availability of the actual data to be assessed. In some examples, the application used to implement the data management circuitry 212 can be designed on top of a cloud computing platform (e.g., Amazon Web Services™ (AWS) platform, etc.). For example, the cloud-computing platform (e.g., AWS™) can include a mixture of infrastructure as a service (IaaS), platform as a service (PaaS) and/or packaged software as a service (SaaS). By using a cloud provider, access to managed services with a range of functionality can be obtained. As such, the process development central data management circuitry 212 can be integrated into existing managed service platforms (e.g., AWS™) instead of just being hosted on the platform and/or used as a stand-alone computer-based software. While the data management circuitry 212 is presented as a stand-alone software based on the descriptions of FIGS. 15-18 below, the data management circuitry 212 described herein can instead be a packaged software as a service (SaaS) (e.g., using the AWS™ platform, etc.).

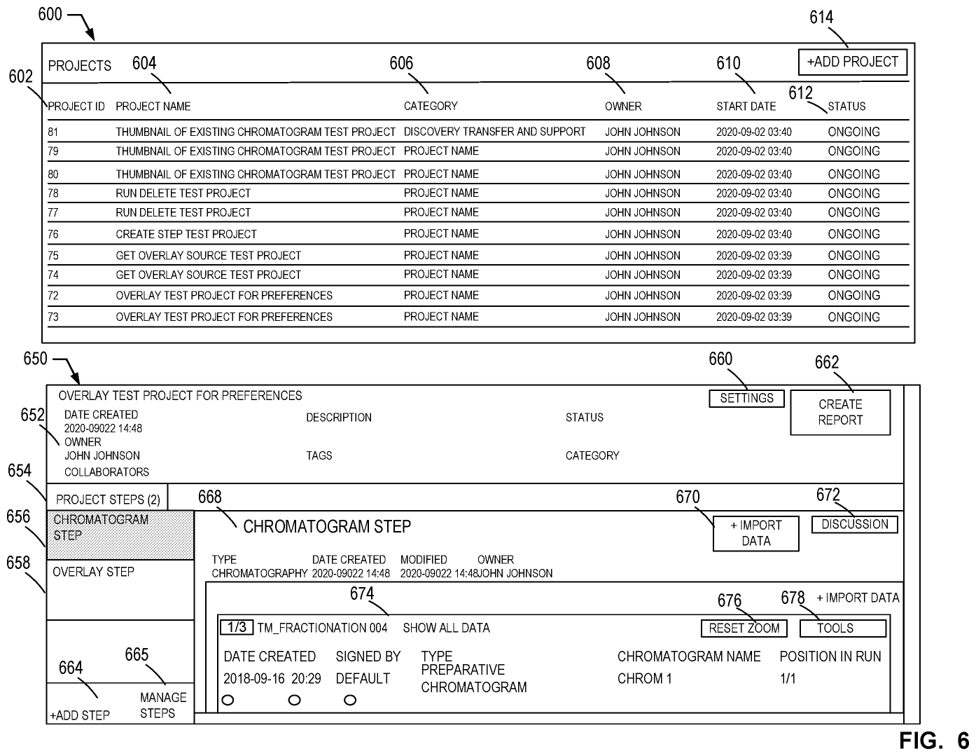

FIG. 5 illustrates an example collection of data for integral data management 500. In the example of FIG. 5, collection of data for integral data management 500 includes a process involving validation of analytical techniques 502, buffer and chemicals preparations 504, column packing and validation 506, cleaning and validation of instrumentation 508, and/or system priming and equilibration 510. In some examples, an electronic laboratory notebook (ELN) 512 can be used to record information related to sample and/or buffer preparation 514, ultrafiltration (UF) and/or diafiltration (DF) 516, chromatography (e.g., anion exchange chromatography (AEX) 516 and/or hydrophobic interaction chromatography (HIC) 520), additional filtration data associated with ultrafiltration and/or diafiltration 522, and final output chromatogram(s) and/or batch report(s) 524. In some examples, the ELN serves as a record of data associated with one or more bioreactor(s) (e.g., a first bioreactor 526, a second bioreactor 528, etc.), including any bioreactor processes (e.g., a first bioreactor run 530, a second bioreactor run 532, etc.). In some examples, the ELN records information associated with any part of an example bioprocess 540 (e.g., high performance liquid chromatography (HPLC) used for separating the components of a compound or mixture, ultraviolet-visible spectroscopy, purity calculation, etc.). In the examples disclosed herein, the central data management circuitry 212 can capture information from ELNs, including additional information such as analytical results, collaboration comments, and any other information associated with the process data. In examples disclosed here, files can be uploaded for purposes of visualization, interaction, and reporting among individuals associated with a given project. Additionally, process data can be integrated with analytical data and results, and further contextualized with metadata. In some examples, experimental data can be organized based on process and/or project steps, with options for generic tagging, annotation, data exporting, and/or reporting. Furthermore, the central data management circuitry 212 can be used to provide sharing and collaboration functionalities, project access control functionalities, and/or user-specific and organization-specific management functionalities, as described below in connection with FIGS. 6-14.

FIG. 6 illustrates example application screen overviews 600, 650 used for creating and editing projects based on the process development central data management circuitry 212 of FIG. 3. In the example of FIG. 6, an application opens on a “Projects” tab by default, listing projects accessible via a process development central data management system based on the data management circuitry 212 of FIG. 2 (e.g., projects corresponding to a specific user's project listing access). The “Projects” tab can likewise be used to create and/or edit project(s). Additionally, the “Reports” tab lists reports that have been created by the user or projects that the user has authorization to access (e.g., projects created by other users, etc.). In some examples, selecting the user name on the right-hand side of the application header can be used to access: (1) a user profile that can be displayed and/or edited, (2) an organization (e.g., name and contact information of an administrator), (3) data management software version information, and/or (4) a log-out selection to exit the system. In the example of FIG. 6, the application screen overview 600 includes an example project ID identifier 602 (e.g., ID of the project), an example project name identifier 604 (e.g., name of the project), an example category identifier 606 (e.g., category of the project), an owner identifier 608 (e.g., an owner of the project), a start date identifier 610 (e.g., a start date of the project), and/or a status identifier 612 (e.g., an ongoing project or an archived project). In some examples, an “Add Projects” option 614 allows a user to initiate a new project. In some examples, once the “Add Projects” option 614 is selected, a user can be prompted to enter information shown in connection with application screen overview 650. For example, the user can be prompted to enter information corresponding to a project name, a project description, a project category, project collaborators, and/or tags associated with the project. The project details can be viewed based on the entered user information, as shown in connection with application screen overview 650, which includes example project details 652, including a date of project creation, owner(s), collaborator(s), description(s), tag(s), project status, and/or project category.

In some examples, a “Project Steps” section 654 includes projects steps (e.g., chromatogram step 656, overlay step 658, etc.), including the options to add additional steps using “Add Step” 664 and/or manage steps using “Manage Steps” 665. In the example of FIG. 6, the project step selected is the chromatogram step 656, which can include project step details such as step name, creation data, date of the last modification, step owner's name, step type, and/or step description (e.g., shown in example chromatogram step view 668). For example, the user can obtain a detailed view of experiments and/or chromatography overlay(s) associated with a given step. Additionally, the detailed view (e.g., chromatogram step view 668) can include recently used chromatography/overlay. Likewise, if analytical data is imported into the chromatogram, the analytical data can be shown below the chromatogram (e.g., data view 674). In some examples, the step type can be programmed to facilitate how a user is working and/or accommodate the activities the user is performing (e.g., the “process step” can include a leading dataset with a tabulated format of data and/or results shown below the given dataset). As such, the organization of the data being shown to a user (e.g., based on a particular process step) facilitates user comprehension of the progress associated with the particular step of the process. Likewise, additional selection options shown in connection with the application screen overview 650 of FIG. 6 can include an example settings selection 660 used to identify and/or alter settings and an example create report selection 662 used to create a report based on the available information. In some examples, every item in the example user interface can be exported to the report. In some examples, as a user builds up a structure in a given project and selects specific focusing views in a given visualization, the report is being built up as the user interacts with the data, thereby reducing the time to generate a report when such an action is requested by the user. For example, information listed by the user in the “description” section can be automatically included with a given step information in the generated report. Other selections can include an example import data selection 670 to select data for importing, an example discussion selection 672 to identify and/or initiate a discussion about an existing project with project collaborators, an example reset zoom selection 676 to reset the zooming setting when viewing data, and/or an example tools selection 678 for accessing available tools for managing the data. Such a process obviates the need for manual creation of a report, which saves time and reduces potential manual errors.

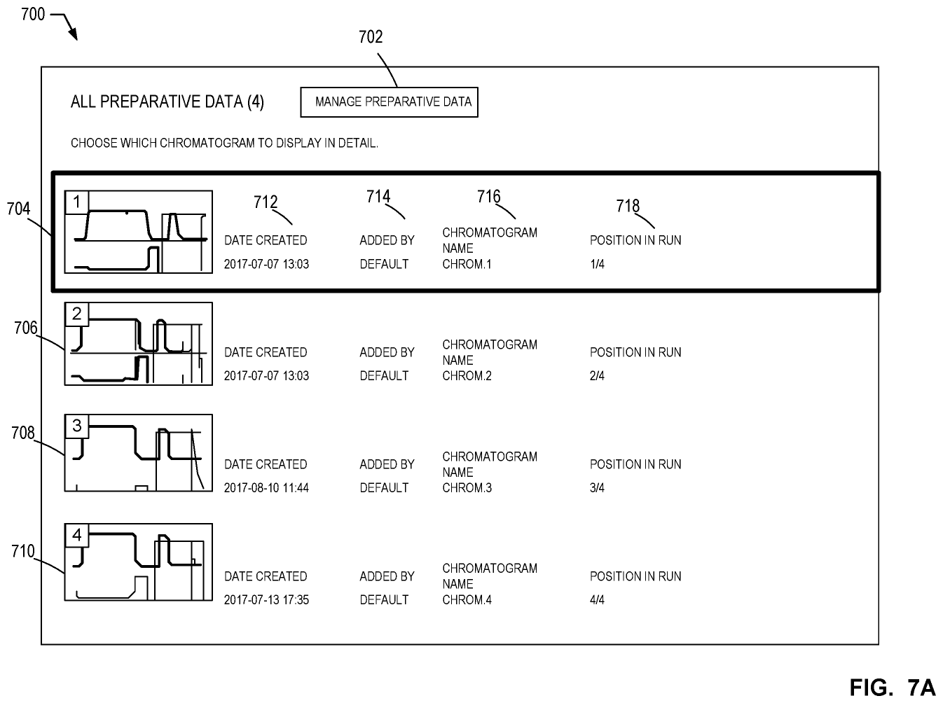

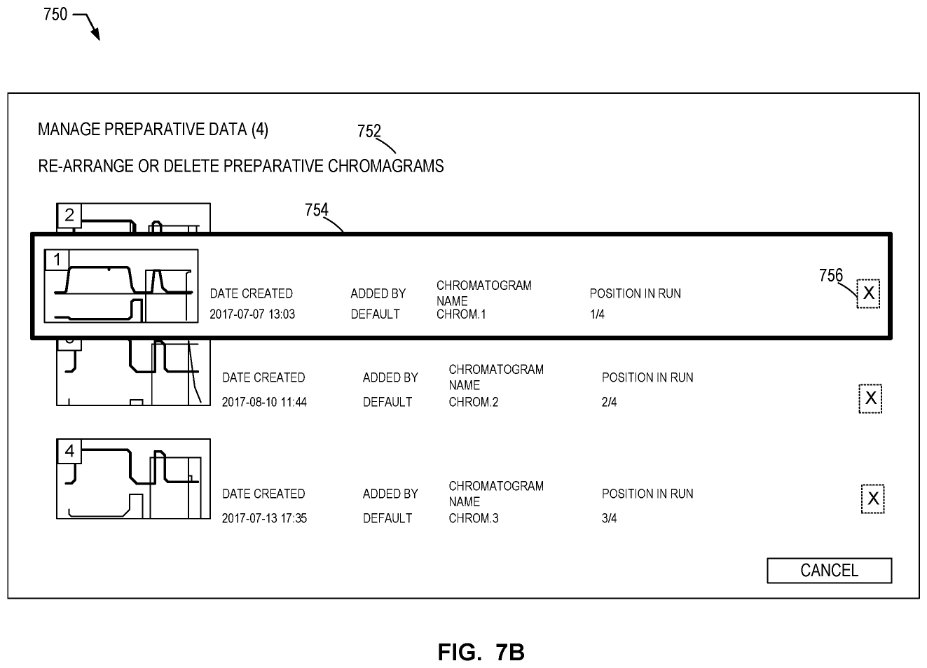

FIG. 7A illustrates an example application screen overview 700 used for viewing and/or managing preparative data and/or creating overlays of existing data associated with the process development central data management circuitry 212 of FIG. 3. In some examples, detailed views associated with the application screen overview 650 of FIG. 6 can also include functions such as “All preparative data/All overlays” to select a chromatogram/overlay other than the one displayed. In the example of FIG. 7A, preparative data can be managed using a manage preparative data selection 702. In some examples, the preparative data can include several chromatogram(s) that can be available for display based on a listing of the chromatograms (e.g., first chromatogram data 704, second chromatogram data 706, third chromatogram data 708, fourth chromatogram data 710, etc.). In some examples, the displayed chromatogram(s) can include information such as the date of creation 712, identification of user providing the data 714, chromatogram name 716, and/or position in a data collection period 718. FIG. 7B illustrates an example application screen overview 750 used for viewing and/or managing preparative data and/or re-arranging preparative chromatograms associated with the process development central data management circuitry 212 of FIG. 3. For example, the application screen overview 750 shows an option to re-arrange and/or delete preparative chromatograms 752. In some examples, a chromatogram selection 754 allows the user to delete the chromatogram data (e.g., using delete option 756) and/or re-arrange the positioning of the data in the order of chromatograms shown in FIG. 7A (e.g., first chromatogram data 704, second chromatogram data 706, third chromatogram data 708, fourth chromatogram data 710, etc.).

FIG. 8A illustrates an example application screen overview 800 used for data pairing associated with the process development central data management circuitry 212 of FIG. 3. For example, analytical data can be paired with preparative chromatogram fractions or fraction pools in a chromatography project step. In some examples, a user can select a project from a list of projects and select a project step that has the preparative chromatogram with analytical data imported into the preparative chromatogram. In some examples, the data can be paired manually by selecting the fraction name followed by the corresponding analytical data. In some examples, a fraction pooling function can be used to pair multiple fractions with a single analytical data item. In the example of FIG. 8A, data pairing is performed by allowing a user to select fraction(s) and analytical data that the user would like to pair with the given fraction. In the example of application screen overview 800, the user can view a fraction name 802, a sample name 804, a result identifier 806, a date of creation 808, and/or an operator name 810. In some examples, the user can select a “pair data” option to initiate the data pairing. Once paired data is created, the paired data can be viewed in a separate section and/or window (e.g., paired data view 812).

FIG. 8B illustrates an example application screen overview 850 used for chromatogram overlays performed in connection with the process development central data management circuitry 212 of FIG. 3. For example, preparative chromatogram curves can be overlayed on each other for comparison purposes. In some examples, the user can select an overlay project step that the user would like to create an overlay for or create a new overlay step (e.g., using a browse steps option 852). In some examples, the user can select one or more chromatograms to add to the overlay. In the example application screen overview 850, the chromatogram(s) of the selected step appear as chromatogram tiles 854, 856, 858. In some examples, additional data related to the chromatograms selected for overlaying can be shown in a separate section 860 of the application screen overview 850. An example “create overlay” selection 862 can be used by the user to initiate the overlay formation using the selected data. In some examples, the user can save a report of the data, including, but not limited to, project information, project steps, all imported data (e.g., preparative/analytical chromatogram charts, corresponding metadata and/or attachment(s), etc.), as well as pairing information. In some examples, user preferences setup by the user (e.g., zoom level, highlighted curves, selected curves, favorites, pairing, etc.) can be applied to the contents of the report.

FIG. 9 illustrates an example application screen overview 900 used for managing process data chromatograms in connection with the process development central data management circuitry 212 of FIG. 3. For example, if a preparative chromatogram is imported into a selected step, the chromatogram appears as shown in application screen overview 900. Several functions can be available for preparative chromatograms (in chromatography steps) and/or overlays (e.g., in overlay steps). For example, curves can be customized (e.g., a user can select which curves are displayed in the chart, change the x-axis base unit to time or to volume, etc.). In some examples, the user can perform zooming functions, displaying of the x and/or y-axes values, highlighting a curve, and/or deleting a chromatogram/overlay. In the example of FIG. 9, the application screen overview 900 includes a preparative data selection 902 to view all preparative data, a previous selection 904 to proceed to the previous chromatogram, a next selection 906 to proceed to the next chromatogram, an import data selection 908 to import data, and/or an add chromatogram description option 910 to add additional information about the chromatogram being viewed. In some examples, a chromatogram view 912 can be magnified and/or minimized using a zoom function, as described above.

FIG. 10 illustrates an example application screen overview 1000 used for downloading process data chromatograms in connection with the process development central data management circuitry 212 of FIG. 3. In some examples, a snapshot of the analytical chromatogram chart from a fraction can be viewed and/or downloaded (e.g., using a portable network graphic (PNG) format, etc.). In the example of FIG. 10, fraction data can be saved with current settings and/or customization (e.g., curves, zoom, legends, etc.) saved with the image. In some examples, a user can identify an area of interest on a given curve and zoom in on that area. The identified area of interest (e.g., zoomed-in area) can be included in the generated report. In the example of FIG. 10, a user can select a download data option 1004 and/or a download chart option 1006 to save the corresponding data shown as part of the application screen overview 1000. In some examples, the user can reset the zoom level using a reset zoom option 1008 (e.g., return to a default view from a minimized view or a maximized view, etc.) of the corresponding chromatogram chart 1010. In some examples, the user can view and/or access basic information 1012 and/or details and results 1014 associated with the chromatogram chart 1010 as part of the application screen overview 1000 and/or as part of the downloaded data/downloaded chart. In some examples, access to the data can be determined based on whether a user has been invited to view the data (e.g., as part of user activation and/or deactivation).

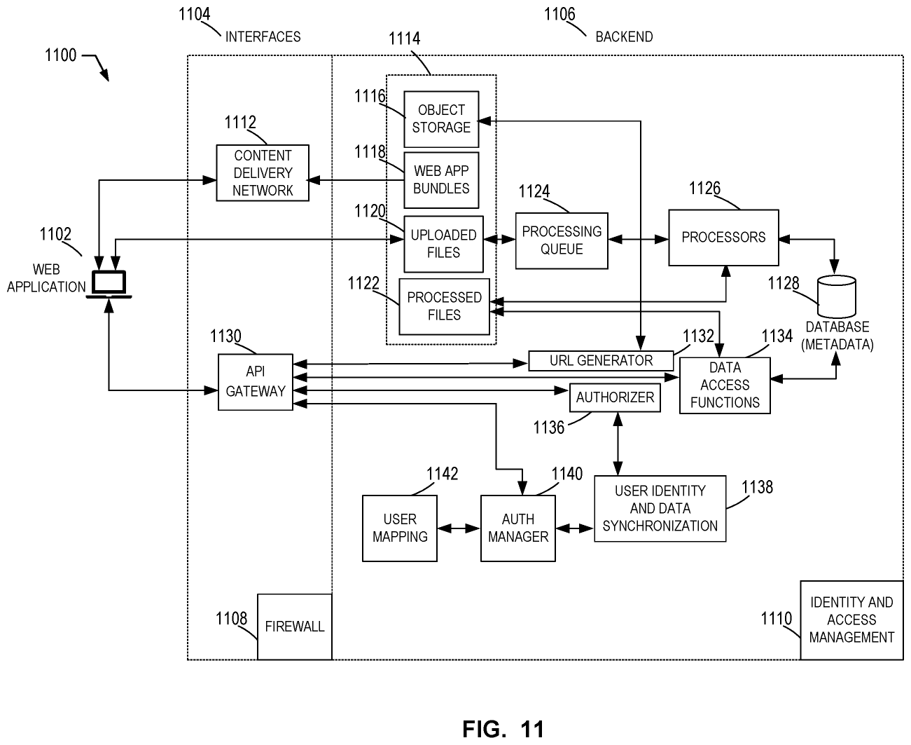

FIG. 11 illustrates an example architecture 1100 implemented in connection with the process development central data management circuitry 212 of FIG. 3. For example, the example architecture 1100 can be used to integrate process data, resulting in generic data and knowledge management and/or search, export, and report generation capabilities. In some examples, data can be obtained from data source(s) and/or applications for managing and/or interacting with process data (e.g., software such as UNICORN® and/or Empower, etc.). As such, methods and apparatus disclosed herein allow for the visualization of process (e.g., preparative) and analytical chromatograms and association of analytical results with process (e.g., preparative) chromatography fractions. However, any other type of visualization and/or association of process data not limited to chromatogram-based data can be performed using the methods and apparatus disclosed herein. In the example of FIG. 11, a web application 1102 can be a single page application hosted on a server (e.g., a cloud computing server such as Amazon Web Services (AWS)™, Inc., etc.). In some examples, the web application 1102 is served through a content delivery network 1112 (e.g., AWS™ CloudFront, etc.) with access restricted to whitelisted addresses. In some examples, the web application 1102 can be accessible using Hypertext Transfer Protocol Secure (HTTPS) (e.g., by applying a Command and Query Responsibility Segregation (CQRS) pattern, etc.). In some examples, the web application 1102 can be developed using a platform for building mobile and/or desktop web applications (e.g., Angular, etc.). In some examples, the web application 1102 can reach backend capabilities (e.g., using an application programming interface (e.g., RESTful API provided by an application programming interface (API) Gateway 1130, etc.)). In the example of FIG. 11, the architecture 1100 includes an interface section 1104 and a backend section 1106. For example, the interface section 1104 includes the content delivery network 1112, the API gateway 1130, and a firewall 1108. In some examples, the backend section 1106 can include cloud computing server services including, but not limited to, a storage service 1114 for object storage 1116, web application bundles 1118, uploaded files 1120, and/or processed files 1122. In some examples, secure cloud services can be used (e.g., Amazon Web Services IM Elastic Compute Cloud (EC2), etc.) and/or any other third-party provided software. In some examples, the services described herein can be implemented using an infrastructure as a service (IaaS)-based solution.

In the example of FIG. 11, backend section 1106 feature endpoints can be managed using the API gateway 1130, with the endpoints providing access to login, filing uploading, obtaining data for a context, and/or pushing data for a context. In the example of FIG. 11, user identity and data synchronization 1138 integrates with the other available services and assists in meeting security requirements by providing access control, standards-based authentication, supporting multiple compliance programs, etc. In some examples, the user identity and data synchronization 1138 can include a cloud-based identity and access management service 1110 (e.g., AWS™ identity and access management (IAM), etc.) to provide roles and associated permissions (e.g., with users mapped to IAM roles, etc.). For example, users can be mapped using user mapping 1142 to grant users access to data (e.g., process data, analytical data, chromatogram-based information, etc.) associated with a project. An authorization manager 1140 can be used to authorize access and is in communication with the user mapping 1142 service and user identity and data synchronization 1138 service. In the example of FIG. 11, the API gateway 1130 manages the authorization manager 1140, a uniform resource locator (URL) generator 1132, a data access functions service 1134, and/or an authorizer 1136, given that the system needs to track data about users and tenants to provide authentication and authorization, with data accessed a predefined number of times (e.g., once per user login, etc.). As such, the data access functions service 1134 is in communication with a database 1128.

In some examples, the database can include a fully managed, serverless database (e.g., AWS™ DynamoDB, AWS™ Aurora Serverless, AWS™ Relational Database Service (RDS), etc.) which can be selected based on a database of interest (e.g., a database using a Structured Query Language (SQL), a database using noy only SQL (NoSQL), a database using multiple different database management systems (DBMS), etc.). The database 1128 can include experiment data (e.g., data derived from uploaded run files such as chromatogram curves, metadata of chromatogram results, peak tables, fraction tables, injections, etc.). For example, as the curve data is different in size and nature, the curve data can be handled separately from all the other attributes. In some examples, the database 1128 includes context data (e.g., data of contextual containers that encapsulate the experimental data such as project, and information about project steps, etc.). In some examples, the database 1128 can include user added data (e.g., all data users add manually, such as fraction associations, comments and tags, etc.). In some examples, the type of database 1128 that can be used can be selected based on a cardinality of entities in the broadest context (e.g. maximum number of rows in a table at a time), query flexibility requirements (how much freedom will users have to query data in different ways), the need for and complexity of data sorting with server-side pagination, complexity of different migration scenarios (e.g., migrate from DynamoDB to RDS and the other way around), cost, and/or support for customer managed keys (e.g., CMKs).

In the example of FIG. 11, the database 1128 is in communication with processor(s) 1126, which are in turn in communication with a processing queue 1124 and/or processed files 1122, allowing file storage and uploading using the uploaded files 1120 service. As previously described, the storage service 1114 allows for object storage 1116, web application bundles 1118, uploaded files 1120, and/or processed files 1122. In some examples, the stored data is protected with encryption, versioning and/or locking, and access can be controlled with the combination of resource-based and/or user policies. In some examples, separate buckets are used for imported run files (e.g., uploaded files 1122), with a processed bucket used for curve data and/or already processed run files (e.g., processed files 1122).

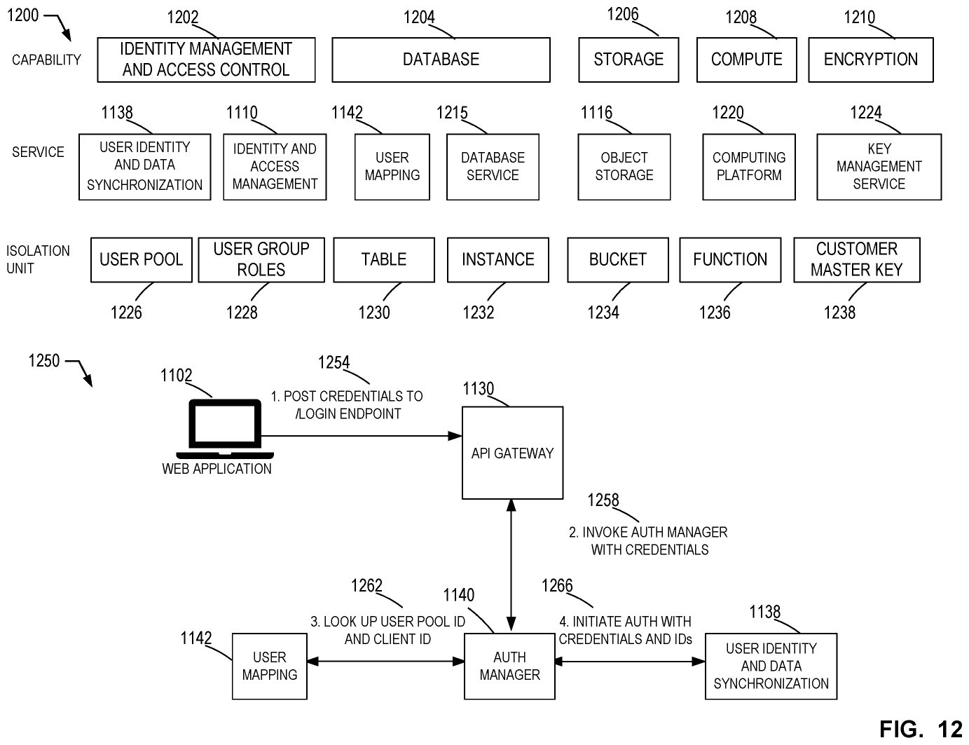

FIG. 12 illustrates an example architecture 1200, 1250 used for logging and monitoring events related to deployment, infrastructure, application, and security in connection with the process development central data management circuitry 212 of FIG. 3. In the example of FIG. 12, an overview is presented of capabilities, services, and isolation units associated with the architecture 1100 of FIG. 11. For example, capabilities can be categorized into identity management and access control 1202, database 1204, storage 1206, compute 1208, and/or encryption 1210. In some examples, services associated with the identity management and access control 1202 capability include user identity and data synchronization 1138 (e.g., AWS™ Cognito, etc.) and identity and access management 1110 (e.g., AWS™ Identity and Access Management, etc.), while isolation units associated with the identity management and access control 1202 capability include user pool(s) 1226 and/or user group role(s) 1228. For example, user pool(s) 1226 can be used for separating the users of different tenants. User pool(s) 1226 can act as separate identity providers such that in the authentication flow a user is authenticated against a user pool, therefore one user in a user pool cannot authenticate using another customer's user pool. In some examples, user group roles(s) 1228 are created with associated identity and access management (IAM) roles per user pool(s) 1226. In some examples, users get mapped to their corresponding IAM roles when calling the API Gateway 1130 of FIG. 11 and permissions are denied or granted based on the policies that are attached to those roles. In some examples, the addition of a new user to the user pool(s) 1226 results in a temporary password being set or generated, such that the user is enforced to change the password during s first login. In some examples, the identification of the tenant the user belongs to is added as a custom attribute of the user pool(s) 1226. The custom attribute can also be added to generated token(s) during authentication as a custom claim to ease the authorization process of subsequent API calls.

In some examples, services associated with the database 1204 capability include user mapping 1142 (e.g., using AWS™ DynamoDB, etc.) and database service 1215 (e.g., AWS™ RDS, etc.), while isolation units associated with the database 1204 capability include table(s) 1230 and/or instance(s) 1232. In some examples, services associated with the storage 1206 capability include object storage 1116 (e.g., using AWS™ S3, etc.), while isolation units associated with the storage 1206 capability include bucket(s) 1234. In some examples, services associated with the compute 1208 capability include computing platform 1220 (e.g., using AWS™ Lambda, etc.), while isolation units associated with the compute 1208 capability include function(s) 1236. For example, computing tasks performed using computing platform 1220 can include Lambda functions. In some examples, Lambda functions can be used to perform authentication, authorization, uploading of URL generation, data processing, and/or create, read, update, delete (CRUD) operations. In some examples, services associated with the encryption 1210 capability include key management service(s) 1224 (e.g., AWS™ Key Management Services, etc.), while isolation units associated with the encryption 1210 capability include customer master key(s) (CMKs) 1238. In some examples, data of different tenants can be encrypted with different tenant-specific keys. In some examples, separate CMKs can be used to encrypt user mapping 1142 tables (e.g., DynamoDB tables, etc.), database service 1215 instances (e.g., Aurora Serverless (RDS) instances, etc.), and/or object storage 1116 buckets (e.g., AWS™ S3 buckets, etc.) of different tenants. In some examples, various services described herein can communicate through encrypted channel(s) using HTTPS. In some examples, encryption in storage and database services can be performed using a fully managed service that allows managing keys and encryption for other services (e.g., AWS™ Key Management Service, etc.).

As described herein, multiple models can be supported in various services for tenant isolation. In some examples, tenants can have access to separate tables or sets of tables in the presence of multiple applications, with one tenant's users only accessing the tables of the same tenant (e.g., table(s) 1230). In some examples, tenants can have separate database instances, such that one tenant's users can only access their instances (e.g., instance(s) 1232). In some examples, tenants can have access to their set of buckets (e.g., bucket(s) 1234), such that one tenant's users can only access their buckets. In some examples, tenants can be isolated with Lambda functions using tenant specific functions and/or tenant agnostic functions. When using tenant specific functions, each function has a tenant specific copy, and the function code can be tenant-aware. In some examples, the functions can be attached to events coming from tenant specific resources (e.g., buckets, tables, etc.). This approach can be used for isolation, but it can be less scalable and managing the function copies can become complex with an increasing number of tenants. When using tenant agnostic functions, function code is not aware of the actual tenant. For example, the necessary context information is passed when the function is invoked, such that a token (e.g., representing a user of a specific tenant) is passed. The token can have claims with the tenant identification or with other custom attributes and the function can derive the context based on the attributes. The function can exchange the token for a temporary credential with assumed IAM roles. This approach can provide tenant isolation without the need of tenant specific code but may not be applicable in all scenarios. Tenant specific customizations can require the use of separate functions. In the examples disclosed herein, tenant agnostic functions are used when possible and tenant specific functions are deployed otherwise. In both cases several versions of the same Lambda function may be deployed and active if some tenants choose not to upgrade to a newer version of the application.

In some examples, the web application 1102 of FIG. 11 can be used to provide a customized login page for the users. The web application 1102 redirects unauthenticated users to the login page. When the user submits the username and password, the web application 1102 calls the login endpoint with the provided credentials to initiate the authentication flow using step 1254 (e.g., posting of credentials and login endpoint, which are provided to the API Gateway 1130). In some examples, a Lambda function is triggered to identify which user pool 1226 to authenticate the user against. Subsequently, the Lambda function can be used to call authentication API and returns with the result. For example, the API Gateway 1130 invokes the Authorization Manager 1140 using the provided credentials at step 1258. The Authorization Manager 1140 initiates the identification of the user pool identifier and/or the client identifier based on the provided credentials (e.g., step 1262) via the user mapping 1142 service. Likewise, the Authorization Manager 1140 initiates authorization with provided credentials and identifiers (e.g., at step 1266) using the user identity and data synchronization 1138 service. If the user is successfully authenticated, obtained tokens are returned to the API Gateway 1130 and back to the web application 1102 in the HTTP response through encrypted channel(s). If the login attempt fails, an error code is sent back to the web application 1102 to notify the user about the failure without exposing details of the error. In some examples, several tokens can be returned after a successful authentication, including an ID token, an access token, and/or a refresh token. In some examples, the refresh token is a long-living token that is not used in the web application 1102, as the refresh mechanism takes place in the backend 1106 of FIG. 11. In some examples, token expiration is handled on the server side as well. In the examples disclosed herein, API calls invoke a custom authorizer (e.g., authorizer 1136) to validate the token with the user pool 1226 of the user's tenant and authorize or deny the request depending on token validity. If the request is authorized, the token is passed in the context object for the Lambda integrated with the called endpoint. In some examples, the system disclosed herein allows for an administrator role (e.g., lab or project managers who will have access to all projects and can assign people to projects) and a user role (e.g., scientists who will use the project management system on daily basis and will have access to the projects they are assigned to). In some examples, groups can be associated with IAM roles which can have multiple policies attached to them. For example, when a function performs a task on behalf of a user, the function assumes the necessary IAM role of that user and temporary credentials are given to the function. Requests made with these temporary credentials are authorized against the permissions granted to the assumed IAM role. In some examples, user actions trigger Lambda functions that can call different services and other functions can be triggered through the events of the involved services. This chain of calls can happen on behalf of a user and the functions obtain IAM permissions based on the role of that user. In some examples, the execution role of the Lambda functions used in these scenarios will not have permissions to use the task-related resources but will be limited to the user-service delegation.

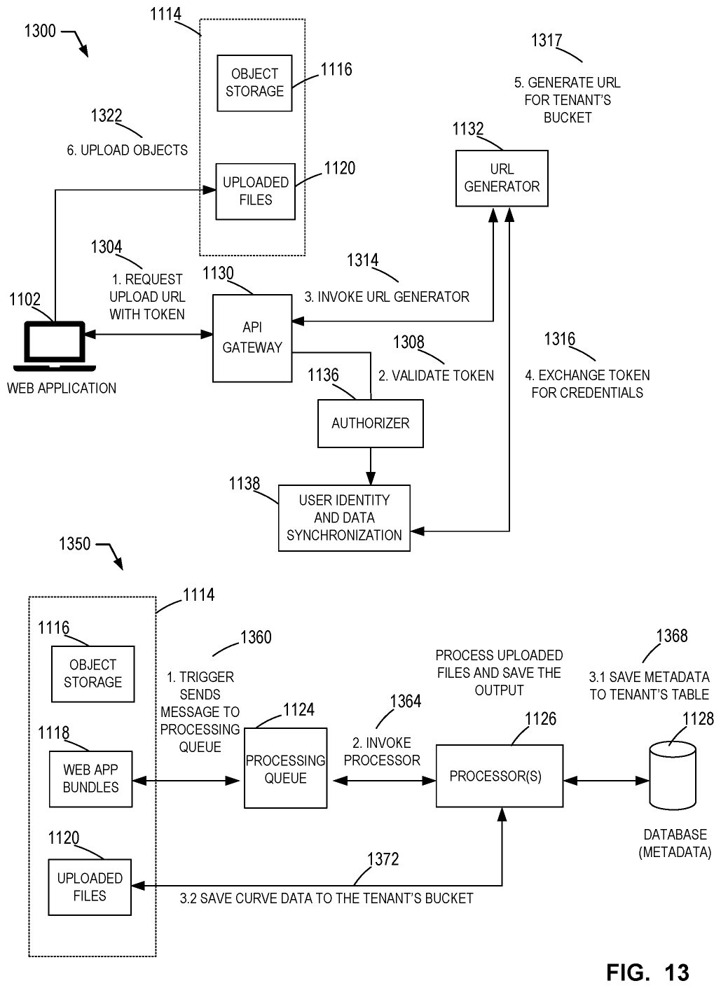

FIG. 13 illustrates an example architecture 1300, 1350 used for uploading files and data processing in connection with the process development central data management circuitry 212 of FIG. 3. In examples disclosed herein, files exported from different instrument software can be uploaded through the web application 1102. The upload can be performed using a pre-signed URL due to API Gateway limitations (e.g., file upload size). For example, such a process can be initiated with an API call to request a URL for uploading to object storage 1116 (e.g., request upload URL with token at step 1304), and web application 1102 can upload the files directly to the object storage 1116 (e.g., by sending a POST request to the obtained URL). In the example of FIG. 13, the API Gateway 1130 is used to validate the token via the authorizer 1136, which initiates user identity and data synchronization using the user identity and data synchronization 1138 service. The URL generator 1132 can be invoked at step 1314. At step 1316, tokens are exchanged for credentials and at step 1317 a URL is generated for the tenant's bucket, followed by object uploading at step 1322 into the storage service 1114, which includes the object storage 1116 and the uploaded files 1120. As such, only authorized users can access the URL and the designated URL can only be used for uploading, while objects cannot be downloaded from the object storage 1116. Using pre-signed URLs with the POST method for upload allows to define numerous conditions for the uploaded object, including name prefix, minimum and maximum size, content type and URL expiry.

In the examples disclosed herein, all received inputs (e.g., user inputs, API call responses, etc.) are validated in the frontend, with the user notified of validation errors. In some examples, a web application firewall (e.g., firewall 1108) is used to monitor web requests on the API Gateway 1130 and filter out malicious requests. In some examples, audit logging can be performed for increased security, including login attempts (e.g., success and failure), authorization failure, data upload (e.g., success and failure), project data changes (e.g., success and failure), and validation failures.

In the example of FIG. 13, the architecture 1350 includes a data uploading process. The data uploading process includes a trigger from the web application bundles 1118 that sends a message to the processing queue 1124 (e.g., at step 1360). The web application bundles 1118 are in the storage service 1114, which includes the object storage 1116 and the uploaded files 1120. The processing queue 1124 invokes the processor(s) 1126 (e.g., at step 1364), which initiate the uploading of the file(s) into the uploaded files 1120 (e.g., saving curve data to the tenant's bucket, etc.) at step 1372. In some examples, the metadata is saved to the tenant's table using the database 1128.

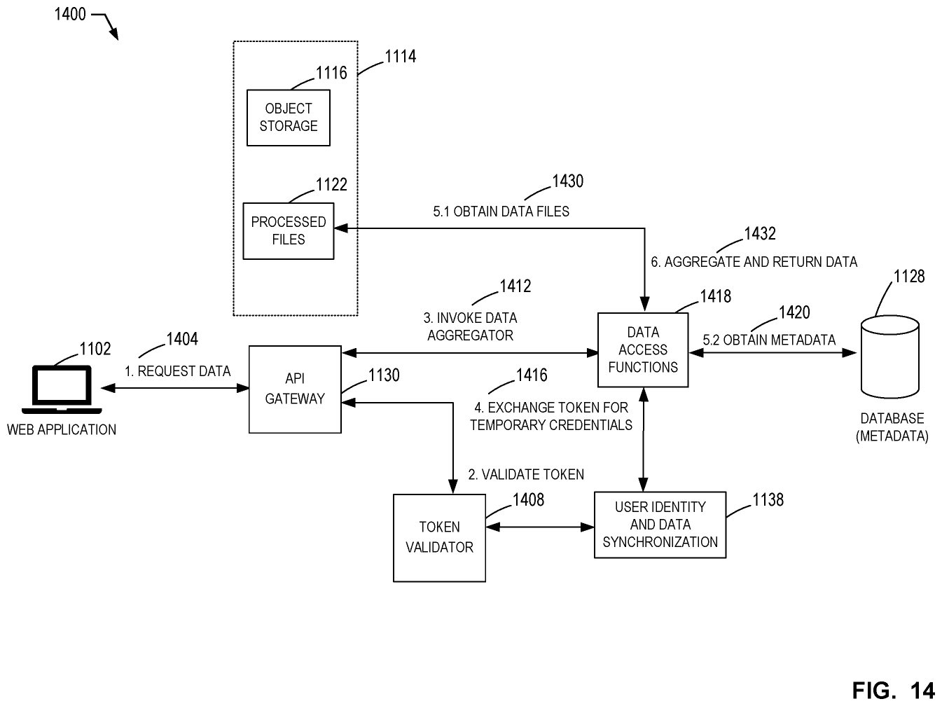

FIG. 14 illustrates an example architecture 1400 used for accessing data in connection with the process development central data management circuitry 212 of FIG. 3. In the example of FIG. 14, the web application 1102 sends a data request (e.g., at step 1404) to the API Gateway 1130. The API Gateway 1130 initiates a process to validate an associated token using a token validator 1408, which can validate the token in connection with the user identity and data synchronization 1138 service. Likewise, the API Gateway 1130 invokes a data aggregator using a Lambda function (e.g., at step 1412) at a data access functions 1418 service, as previously described. In some examples, the data access functions 1418 service exchanges tokens for temporary credentials using the user identity and data synchronization 1138 service (e.g., at step 1416). Once the temporary credentials are obtained, the data access functions 1418 service obtains data file(s) from the processed files 1122 of the storage service 1114 (e.g., at step 1430) and obtains metadata from the database 1128 (e.g., at step 1420). In some examples, the data access functions 1418 service aggregates and returns the data to the processed files 1122. As such, the user can access data using the example architecture 1400.

FIG. 15 is a block diagram of an example processor platform 1500 structured to execute and/or instantiate the machine readable instructions and/or operations of FIG. 4 to implement the process development central circuitry 212 of FIG. 3. The processor platform 1500 can be, for example, a server, a personal computer, a workstation, a self-learning machine (e.g., a neural network), a mobile device (e.g., a cell phone, a smart phone, a tablet such as an iPad™), a personal digital assistant (PDA), an Internet appliance, a DVD player, a CD player, a digital video recorder, a Blu-ray player, a gaming console, a personal video recorder, a set top box, a headset (e.g., an augmented reality (AR) headset, a virtual reality (VR) headset, etc.) or other wearable device, or any other type of computing device.

The processor platform 1500 of the illustrated example includes processor circuitry 1512. The processor circuitry 1512 of the illustrated example is hardware. For example, the processor circuitry 1512 can be implemented by one or more integrated circuits, logic circuits, FPGAs microprocessors, CPUs, GPUs, DSPs, and/or microcontrollers from any desired family or manufacturer. The processor circuitry 1512 may be implemented by one or more semiconductor based (e.g., silicon based) devices. In this example, the processor circuitry 1512 implements the controller circuitry 305, retriever circuitry 310, analyzer circuitry 315, organizer circuitry 320, linker circuitry 325, customizer circuitry 330, viewer circuitry 335, and/or report generator circuitry 340.

The processor circuitry 1512 of the illustrated example includes a local memory 1513 (e.g., a cache, registers, etc.). The processor circuitry 1512 of the illustrated example is in communication with a main memory including a volatile memory 1514 and a non-volatile memory 1516 by a bus 1518. The volatile memory 1514 may be implemented by Synchronous Dynamic Random Access Memory (SDRAM), Dynamic Random Access Memory (DRAM), RAMBUS® Dynamic Random Access Memory (RDRAM®), and/or any other type of RAM device. The non-volatile memory 1516 may be implemented by flash memory and/or any other desired type of memory device. Access to the main memory 1514, 1516 of the illustrated example is controlled by a memory controller 1517.

The processor platform 1500 of the illustrated example also includes interface circuitry 1520. The interface circuitry 1520 may be implemented by hardware in accordance with any type of interface standard, such as an Ethernet interface, a universal serial bus (USB) interface, a Bluetooth® interface, a near field communication (NFC) interface, a PCI interface, and/or a PCIe interface.

In the illustrated example, one or more input devices 122 are connected to the interface circuitry 1520. The input device(s) 1522 permit(s) a user to enter data and/or commands into the processor circuitry 1512. The input device(s) 1522 can be implemented by, for example, an audio sensor, a microphone, a camera (still or video), a keyboard, a button, a mouse, a touchscreen, a track-pad, a trackball, an isopoint device, and/or a voice recognition system.

One or more output devices 1524 are also connected to the interface circuitry 1520 of the illustrated example. The output devices 1524 can be implemented, for example, by display devices (e.g., a light emitting diode (LED), an organic light emitting diode (OLED), a liquid crystal display (LCD), a cathode ray tube (CRT) display, an in-place switching (IPS) display, a touchscreen, etc.), a tactile output device, a printer, and/or speaker. The interface circuitry 1520 of the illustrated example, thus, typically includes a graphics driver card, a graphics driver chip, and/or graphics processor circuitry such as a GPU.

The interface circuitry 1520 of the illustrated example also includes a communication device such as a transmitter, a receiver, a transceiver, a modem, a residential gateway, a wireless access point, and/or a network interface to facilitate exchange of data with external machines (e.g., computing devices of any kind) by a network 1526. The communication can be by, for example, an Ethernet connection, a digital subscriber line (DSL) connection, a telephone line connection, a coaxial cable system, a satellite system, a line-of-site wireless system, a cellular telephone system, an optical connection, etc.

The processor platform 1500 of the illustrated example also includes one or more mass storage devices 1528 to store software and/or data. Examples of such mass storage devices 1528 include magnetic storage devices, optical storage devices, floppy disk drives, HDDs, CDs, Blu-ray disk drives, redundant array of independent disks (RAID) systems, solid state storage devices such as flash memory devices, and DVD drives.

The machine executable instructions 1532, which may be implemented by the machine readable instructions of FIG. 4, may be stored in the mass storage device 1528, in the volatile memory 1514, in the non-volatile memory 1516, and/or on a removable non-transitory computer readable storage medium such as a CD or DVD.

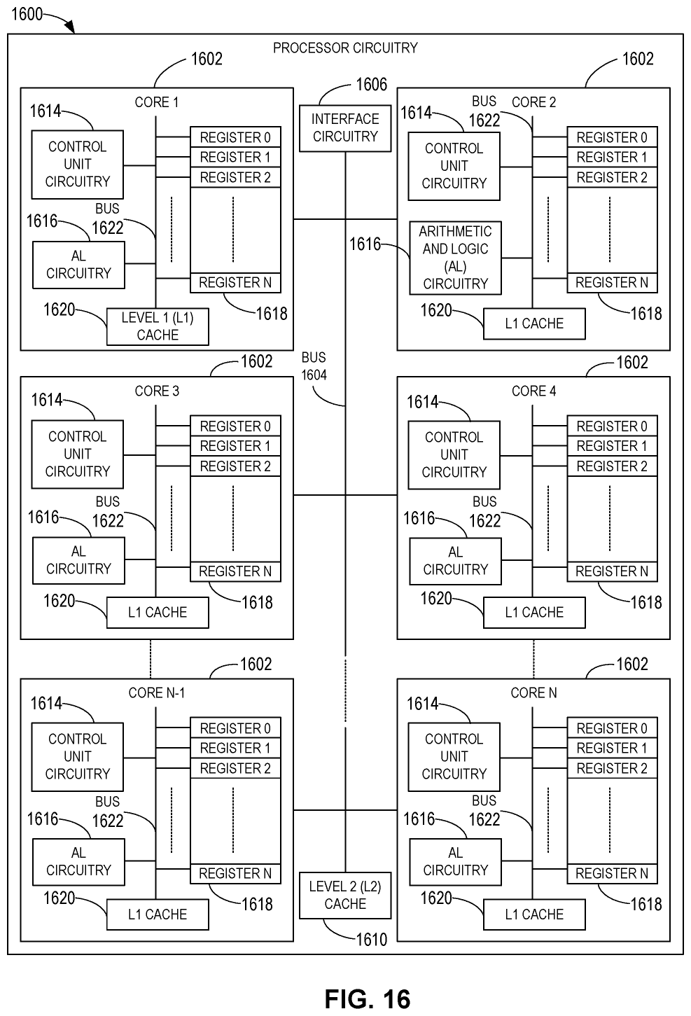

FIG. 16 is a block diagram of an example implementation of the processor circuitry 1512 of FIG. 15. In this example, the processor circuitry 1512 of FIG. 15 is implemented by a microprocessor 1600. For example, the microprocessor 1600 may implement multi-core hardware circuitry such as a CPU, a DSP, a GPU, an XPU, etc. Although it may include any number of example cores 1602 (e.g., 1 core), the microprocessor 1600 of this example is a multi-core semiconductor device including N cores. The cores 1602 of the microprocessor 1600 may operate independently or may cooperate to execute machine readable instructions. For example, machine code corresponding to a firmware program, an embedded software program, or a software program may be executed by one of the cores 1602 or may be executed by multiple ones of the cores 1602 at the same or different times. In some examples, the machine code corresponding to the firmware program, the embedded software program, or the software program is split into threads and executed in parallel by two or more of the cores 1602. The software program may correspond to a portion or all of the machine readable instructions and/or operations represented by the flowchart of FIG. 4.

The cores 1602 may communicate by an example bus 1604. In some examples, the bus 1604 may implement a communication bus to effectuate communication associated with one(s) of the cores 1602. For example, the bus 1604 may implement at least one of an Inter-Integrated Circuit (I2C) bus, a Serial Peripheral Interface (SPI) bus, a PCI bus, or a PCIe bus. Additionally or alternatively, the bus 1604 may implement any other type of computing or electrical bus. The cores 1602 may obtain data, instructions, and/or signals from one or more external devices by example interface circuitry 1606. The cores 1602 may output data, instructions, and/or signals to the one or more external devices by the interface circuitry 1606. Although the cores 1602 of this example include example local memory 1620 (e.g., Level 1 (L1) cache that may be split into an L1 data cache and an L1 instruction cache), the microprocessor 1600 also includes example shared memory 1610 that may be shared by the cores (e.g., Level 2 (L2_ cache)) for high-speed access to data and/or instructions. Data and/or instructions may be transferred (e.g., shared) by writing to and/or reading from the shared memory 1610. The local memory 1620 of each of the cores 1602 and the shared memory 1610 may be part of a hierarchy of storage devices including multiple levels of cache memory and the main memory (e.g., the main memory 1514, 1516 of FIG. 15). Typically, higher levels of memory in the hierarchy exhibit lower access time and have smaller storage capacity than lower levels of memory. Changes in the various levels of the cache hierarchy are managed (e.g., coordinated) by a cache coherency policy.

Each core 1602 may be referred to as a CPU, DSP, GPU, etc., or any other type of hardware circuitry. Each core 1602 includes control unit circuitry 1614, arithmetic and logic (AL) circuitry (sometimes referred to as an ALU) 1616, a plurality of registers 1618, the L1 cache 1620, and an example bus 1622. Other structures may be present. For example, each core 1602 may include vector unit circuitry, single instruction multiple data (SIMD) unit circuitry, load/store unit (LSU) circuitry, branch/jump unit circuitry, floating-point unit (FPU) circuitry, etc. The control unit circuitry 1614 includes semiconductor-based circuits structured to control (e.g., coordinate) data movement within the corresponding core 1602. The AL circuitry 1616 includes semiconductor-based circuits structured to perform one or more mathematic and/or logic operations on the data within the corresponding core 1602. The AL circuitry 1616 of some examples performs integer based operations. In other examples, the AL circuitry 1616 also performs floating point operations. In yet other examples, the AL circuitry 1616 may include first AL circuitry that performs integer based operations and second AL circuitry that performs floating point operations. In some examples, the AL circuitry 1616 may be referred to as an Arithmetic Logic Unit (ALU). The registers 1618 are semiconductor-based structures to store data and/or instructions such as results of one or more of the operations performed by the AL circuitry 1616 of the corresponding core 1602. For example, the registers 1618 may include vector register(s), SIMD register(s), general purpose register(s), flag register(s), segment register(s), machine specific register(s), instruction pointer register(s), control register(s), debug register(s), memory management register(s), machine check register(s), etc. The registers 1618 may be arranged in a bank as shown in FIG. 16. Alternatively, the registers 1618 may be organized in any other arrangement, format, or structure including distributed throughout the core 1602 to shorten access time. The bus 1620 may implement at least one of an I2C bus, a SPI bus, a PCI bus, or a PCIe bus.

Each core 1602 and/or, more generally, the microprocessor 1600 may include additional and/or alternate structures to those shown and described above. For example, one or more clock circuits, one or more power supplies, one or more power gates, one or more cache home agents (CHAs), one or more converged/common mesh stops (CMSs), one or more shifters (e.g., barrel shifter(s)) and/or other circuitry may be present. The microprocessor 1600 is a semiconductor device fabricated to include many transistors interconnected to implement the structures described above in one or more integrated circuits (ICs) contained in one or more packages. The processor circuitry may include and/or cooperate with one or more accelerators. In some examples, accelerators are implemented by logic circuitry to perform certain tasks more quickly and/or efficiently than can be done by a general purpose processor. Examples of accelerators include ASICs and FPGAs such as those discussed herein. A GPU or other programmable device can also be an accelerator. Accelerators may be on-board the processor circuitry, in the same chip package as the processor circuitry and/or in one or more separate packages from the processor circuitry.

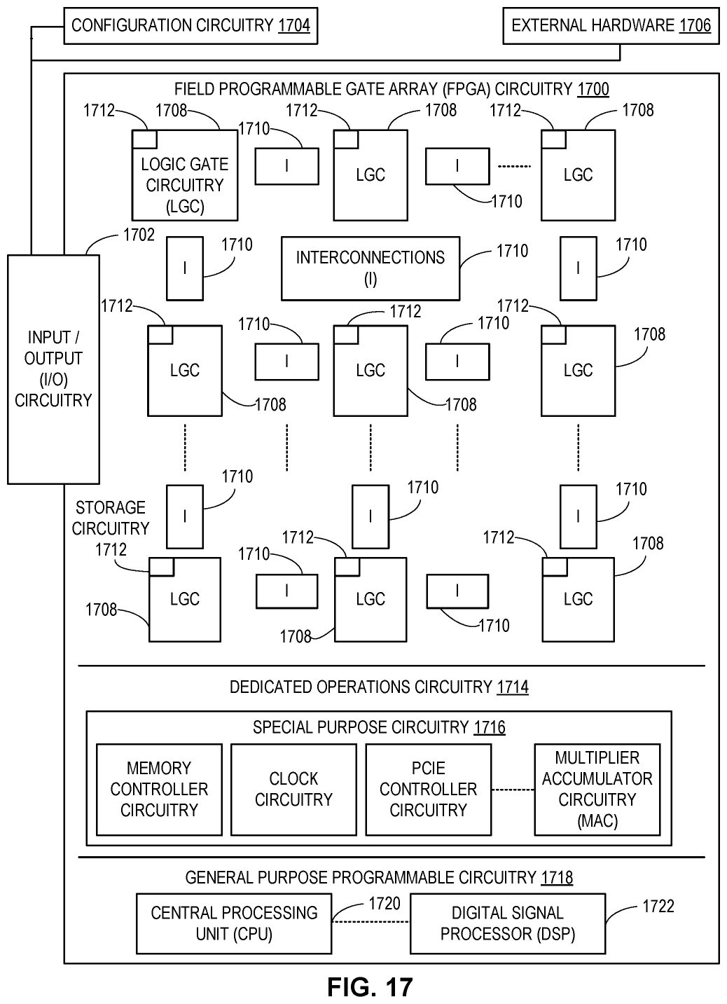

FIG. 17 is a block diagram of another example implementation of the processor circuitry 1512 of FIG. 15. In this example, the processor circuitry 1512 is implemented by FPGA circuitry 1700. The FPGA circuitry 1700 can be used, for example, to perform operations that could otherwise be performed by the example microprocessor 1600 of FIG. 16 executing corresponding machine readable instructions. However, once configured, the FPGA circuitry 1700 instantiates the machine readable instructions in hardware and, thus, can often execute the operations faster than they could be performed by a general purpose microprocessor executing the corresponding software.

More specifically, in contrast to the microprocessor 1600 of FIG. 16 described above (which is a general purpose device that may be programmed to execute some or all of the machine readable instructions represented by the flowchart of FIG. 4 but whose interconnections and logic circuitry are fixed once fabricated), the FPGA circuitry 1700 of the example of FIG. 17 includes interconnections and logic circuitry that may be configured and/or interconnected in different ways after fabrication to instantiate, for example, some or all of the machine readable instructions represented by the flowchart of FIG. 4. In particular, the FPGA 1700 may be thought of as an array of logic gates, interconnections, and switches. The switches can be programmed to change how the logic gates are interconnected by the interconnections, effectively forming one or more dedicated logic circuits (unless and until the FPGA circuitry 1700 is reprogrammed). The configured logic circuits enable the logic gates to cooperate in different ways to perform different operations on data received by input circuitry. Those operations may correspond to some or all of the software represented by the flowchart of FIG. 4. As such, the FPGA circuitry 1700 may be structured to effectively instantiate some or all of the machine readable instructions of the flowchart of FIG. 4 as dedicated logic circuits to perform the operations corresponding to those software instructions in a dedicated manner analogous to an ASIC. Therefore, the FPGA circuitry 1700 may perform the operations corresponding to the some or all of the machine readable instructions of FIG. 4 faster than the general purpose microprocessor can execute the same.

In the example of FIG. 17, the FPGA circuitry 1700 is structured to be programmed (and/or reprogrammed one or more times) by an end user by a hardware description language (HDL) such as Verilog. The FPGA circuitry 1700 of FIG. 17, includes example input/output (I/O) circuitry 1702 to obtain and/or output data to/from example configuration circuitry 1704 and/or external hardware (e.g., external hardware circuitry) 1706. For example, the configuration circuitry 1704 may implement interface circuitry that may obtain machine readable instructions to configure the FPGA circuitry 1700, or portion(s) thereof. In some such examples, the configuration circuitry 1704 may obtain the machine readable instructions from a user, a machine (e.g., hardware circuitry (e.g., programmed or dedicated circuitry) that may implement an Artificial Intelligence/Machine Learning (AI/ML) model to generate the instructions), etc. In some examples, the external hardware 1706 may implement the microprocessor 1600 of FIG. 16. The FPGA circuitry 1700 also includes an array of example logic gate circuitry 1708, a plurality of example configurable interconnections 1710, and example storage circuitry 1712. The logic gate circuitry 1708 and interconnections 1710 are configurable to instantiate one or more operations that may correspond to at least some of the machine readable instructions of FIG. 4 and/or other desired operations. The logic gate circuitry 1708 shown in FIG. 17 is fabricated in groups or blocks. Each block includes semiconductor-based electrical structures that may be configured into logic circuits. In some examples, the electrical structures include logic gates (e.g., And gates, Or gates, Nor gates, etc.) that provide basic building blocks for logic circuits. Electrically controllable switches (e.g., transistors) are present within each of the logic gate circuitry 1708 to enable configuration of the electrical structures and/or the logic gates to form circuits to perform desired operations. The logic gate circuitry 1708 may include other electrical structures such as look-up tables (LUTs), registers (e.g., flip-flops or latches), multiplexers, etc.

The interconnections 1710 of the illustrated example are conductive pathways, traces, vias, or the like that may include electrically controllable switches (e.g., transistors) whose state can be changed by programming (e.g., using an HDL instruction language) to activate or deactivate one or more connections between one or more of the logic gate circuitry 1708 to program desired logic circuits.

The storage circuitry 1712 of the illustrated example is structured to store result(s) of the one or more of the operations performed by corresponding logic gates. The storage circuitry 1712 may be implemented by registers or the like. In the illustrated example, the storage circuitry 1712 is distributed amongst the logic gate circuitry 1708 to facilitate access and increase execution speed.

The example FPGA circuitry 1700 of FIG. 17 also includes example Dedicated Operations Circuitry 1714. In this example, the Dedicated Operations Circuitry 1714 includes special purpose circuitry 1716 that may be invoked to implement commonly used functions to avoid the need to program those functions in the field. Examples of such special purpose circuitry 1716 include memory (e.g., DRAM) controller circuitry, PCIe controller circuitry, clock circuitry, transceiver circuitry, memory, and multiplier-accumulator circuitry. Other types of special purpose circuitry may be present. In some examples, the FPGA circuitry 1700 may also include example general purpose programmable circuitry 1718 such as an example CPU 1720 and/or an example DSP 1722. Other general purpose programmable circuitry 1718 may additionally or alternatively be present such as a GPU, an XPU, etc., that can be programmed to perform other operations.

Although FIGS. 16 and 17 illustrate two example implementations of the processor circuitry 1512 of FIG. 15, many other approaches are contemplated. For example, as mentioned above, modern FPGA circuitry may include an on-board CPU, such as one or more of the example CPU 1720 of FIG. 17. Therefore, the processor circuitry 1512 of FIG. 15 may additionally be implemented by combining the example microprocessor 1600 of FIG. 16 and the example FPGA circuitry 1700 of FIG. 17. In some such hybrid examples, a first portion of the machine readable instructions represented by the flowchart of FIG. 4 may be executed by one or more of the cores 1602 of FIG. 16 and a second portion of the machine readable instructions represented by the flowchart of FIG. 4 may be executed by the FPGA circuitry 1700 of FIG. 17.

In some examples, the processor circuitry 1512 of FIG. 15 may be in one or more packages. For example, the processor circuitry 1600 of FIG. 16 and/or the FPGA circuitry 1700 of FIG. 17 may be in one or more packages. In some examples, an XPU may be implemented by the processor circuitry 1512 of FIG. 15, which may be in one or more packages. For example, the XPU may include a CPU in one package, a DSP in another package, a GPU in yet another package, and an FPGA in still yet another package.

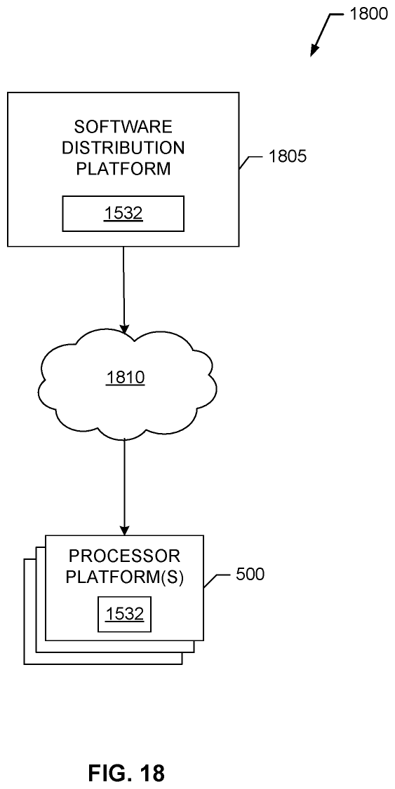

A block diagram illustrating an example software distribution platform 1805 to distribute software such as the example machine readable instructions 1532 of FIG. 15 to hardware devices owned and/or operated by third parties is illustrated in FIG. 18. The example software distribution platform 1805 may be implemented by any computer server, data facility, cloud service, etc., capable of storing and transmitting software to other computing devices. The third parties may be customers of the entity owning and/or operating the software distribution platform 1805. For example, the entity that owns and/or operates the software distribution platform 1805 may be a developer, a seller, and/or a licensor of software such as the example machine readable instructions 1532 of FIG. 15. The third parties may be consumers, users, retailers, OEMs, etc., who purchase and/or license the software for use and/or re-sale and/or sub-licensing. In the illustrated example, the software distribution platform 1805 includes one or more servers and one or more storage devices. The storage devices store the machine readable instructions 1532, which may correspond to the example machine readable instructions 400 of FIG. 4, as described above. The one or more servers of the example software distribution platform 1805 are in communication with a network 1810, which may correspond to any one or more of the Internet and/or any of the example networks described above. In some examples, the one or more servers are responsive to requests to transmit the software to a requesting party as part of a commercial transaction. Payment for the delivery, sale, and/or license of the software may be handled by the one or more servers of the software distribution platform and/or by a third party payment entity. The servers enable purchasers and/or licensors to download the machine readable instructions 1532 from the software distribution platform 1805. For example, the software, which may correspond to the example machine readable instructions 400 of FIG. 4, may be downloaded to the example processor platform 1500, which is to execute the machine readable instructions 1532 to implement the process development central circuitry 212. In some example, one or more servers of the software distribution platform 1805 periodically offer, transmit, and/or force updates to the software (e.g., the example machine readable instructions 1532 of FIG. 15) to ensure improvements, patches, updates, etc., are distributed and applied to the software at the end user devices. In some examples, the software distribution platform 1805 implements the architecture(s) 1100, 1200, 1250, 1300, 1350, 1400, and/or 1500 of FIGS. 11, 12, 13, 14, and/or 15. For example, the software distribution platform 1805 can be used to allow user access to various feature(s) and/or application(s) of the process development central data management circuitry 212 of FIGS. 2 and/or 3. For example, the software distribution platform 1805 can be used to allow a user to link information from electronic laboratory notebook(s) (ELNs) or from laboratory devices to an existing project, which enables the user to access feature(s) that link process-based information from across different device(s) and/or platforms to collaborate and/or share data corresponding to specific process-related projects (e.g., compile data to form overlayed chromatograms, link data (e.g., relevant gel images) obtained from one user to a project initiated by another user, etc.).

From the foregoing, it will be appreciated that the above disclosed methods, apparatus, and articles of manufacture permit improved processing data organization and reporting. For example, methods and apparatus described herein permit the implementation of secure, cloud-based software that represents a single platform with a centralized database for collecting, integrating, visualizing and reporting all data relevant to a given process and/or user. Furthermore, the examples disclosed herein permit integrated data analysis and assessment, streamlined data reporting, and a platform-wide, accessible database of stored results. Methods and apparatus disclosed herein reduce the time for data processing and/or analysis, increase data integrity and traceability, and secure data for re-use across an entire organization. In the examples disclosed herein, a process development central software tool can collect data from different process (e.g., science and analytical) lab devices in upstream and/or downstream process development.

Example methods and apparatus for cloud-based data management and reporting for bioprocess development are disclosed herein. Further examples and combinations thereof include the following:

Example 1 includes a system for process data management, the system comprising memory, and processor circuitry to execute machine readable instructions to at least retrieve a first data set from at least one of an electronic laboratory notebook or a first laboratory device, retrieve a second data set from a second laboratory device, link the first data set and the second data set to a project, the first data and the second data set linked to the project based on a type of data in the first data set and the second data set, generate a data overlay using the first data set and the second data set, the data overlay including an overlay of a first chromatogram and a second chromatogram, generate a report including the data overlay, the data overlay a result of a bioprocessing step associated with the project, link the report to the project, the project accessible to at least two users associated with the project, and track changes made by the at least two users to the first data set or the second data set associated with the report.

Example 2 includes the system of example 1, wherein the electronic laboratory notebook or the laboratory device receives the report, the report including an integration of a first data file in parallel with a second data file.

Example 3 includes the system of example 1, wherein the processor circuitry is to link the first data set to a project to make the first data set searchable in relation to the project.

Example 4 includes the system of example 1, further including processor circuitry to negotiate at least one parameter between the system for process data management and a data source system.

Example 5 includes the system of example 4, wherein the at least one parameter includes an information transfer rate or an interrupt procedure.

Example 6 includes the system of example 1, further including processor circuitry to modify the first data set or the second data set data based on a desired assessment or a type of data output, the type of data output a graphical data output or a tabulated data output.

Example 7 includes the system of example 1, further including processor circuitry to link the first data set to the second data set based on a user-based search entry, the first data set and the second data set a same data type, the data type including a graphical data type or a tabulated data type.

Example 8 includes a method for process data management, the method comprising retrieving a first data set from at least one of an electronic laboratory notebook or a first laboratory device, retrieving a second data set from a second laboratory device, linking the first data set and the second data set to a project, the first data and the second data set linked to the project based on a type of data in the first data set and the second data set, generating a data overlay using the first data set and the second data set, the data overlay including an overlay of a first chromatogram and a second chromatogram, generating a report including the data overlay, the data overlay a result of a bioprocessing step associated with the project, linking the report to the project, the project accessible to at least two users associated with the project, and tracking changes made by the at least two users to the first data set or the second data set associated with the report.

Example 9 includes the method of example 8, wherein the electronic laboratory notebook or the laboratory device receives the report, the report including an integration of a first data file in parallel with a second data file.

Example 10 includes the method of example 8, further including linking the first data set to a project to make the first data set searchable in relation to the project.

Example 11 includes the method of example 8, further including negotiating at least one parameter between the system for process data management and a data source system.

Example 12 includes the method of example 11, wherein the at least one parameter includes an information transfer rate or an interrupt procedure.

Example 13 includes the method of example 8, further including modifying the first data set or the second data set data based on a desired assessment or a type of data output, the type of data output a graphical data output or a tabulated data output.

Example 14 includes the method of example 8, further including linking the first data set to the second data set based on a user-based search entry, the first data set and the second data set a same data type, the data type including a graphical data type or a tabulated data type.

Example 15 includes At least one computer readable storage medium comprising instructions that, when executed, cause at least one processor to at least retrieve a first data set from at least one of an electronic laboratory notebook or a first laboratory device, retrieve a second data set from a second laboratory device, link the first data set and the second data set to a project, the first data and the second data set linked to the project based on a type of data in the first data set and the second data set, generate a data overlay using the first data set and the second data set, the data overlay including an overlay of a first chromatogram and a second chromatogram, generate a report including the data overlay, the data overlay a result of a bioprocessing step associated with the project, link the report to the project, the project accessible to at least two users associated with the project, and track changes made by the at least two users to the first data set or the second data set associated with the report.

Example 16 includes the at least one storage medium as defined in example 15, wherein the computer readable instructions, when executed, cause the one or more processors to negotiate at least one parameter between the system for process data management and a data source system.

Example 17 includes the at least one storage medium as defined in example 15, wherein the computer readable instructions, when executed, cause the one or more processors to modify the first data set or the second data set data based on a desired assessment or a type of data output, the type of data output a graphical data output or a tabulated data output.

Example 18 includes the at least one storage medium as defined in example 15, wherein the computer readable instructions, when executed, cause the one or more processors to link the first data set to the second data set based on a user-based search entry, the first data set and the second data set a same data type, the data type including a graphical data type or a tabulated data type.

Example 19 includes the at least one storage medium as defined in example 15, wherein the electronic laboratory notebook or the laboratory device receives the report, the report including an integration of a first data file in parallel with a second data file.

Example 20 includes the at least one storage medium as defined in example 15, wherein the computer readable instructions, when executed, cause the one or more processors link the first data set to a project to make the first data set searchable in relation to the project.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. A system for process data management, the system comprising:
memory; and
processor circuitry to execute machine readable instructions to at least:
retrieve a first data set from at least one of an electronic laboratory notebook or a first laboratory device;
retrieve a second data set from a second laboratory device;
link the first data set and the second data set to a project, the first data and the second data set linked to the project based on a type of data in the first data set and the second data set;
generate a data overlay using the first data set and the second data set, the data overlay including an overlay of a first chromatogram and a second chromatogram;
generate a report including the data overlay, the data overlay a result of a bioprocessing step associated with the project;
link the report to the project, the project accessible to at least two users associated with the project; and
track changes made by the at least two users to the first data set or the second data set associated with the report.

2. The system of claim 1, wherein the electronic laboratory notebook or the laboratory device receives the report, the report including an integration of a first data file in parallel with a second data file.

3. The system of claim 1, wherein the processor circuitry is to link the first data set to a project to make the first data set searchable in relation to the project.

4. The system of claim 1, further including processor circuitry to negotiate at least one parameter between the system for process data management and a data source system.

5. The system of claim 4, wherein the at least one parameter includes an information transfer rate or an interrupt procedure.

6. The system of claim 1, further including processor circuitry to modify the first data set or the second data set data based on a desired assessment or a type of data output, the type of data output a graphical data output or a tabulated data output.

7. The system of claim 1, further including processor circuitry to link the first data set to the second data set based on a user-based search entry, the first data set and the second data set a same data type, the data type including a graphical data type or a tabulated data type.

8. A method for process data management, the method comprising:
retrieving a first data set from at least one of an electronic laboratory notebook or a first laboratory device;
retrieving a second data set from a second laboratory device;
linking the first data set and the second data set to a project, the first data and the second data set linked to the project based on a type of data in the first data set and the second data set;
generating a data overlay using the first data set and the second data set, the data overlay including an overlay of a first chromatogram and a second chromatogram;
generating a report including the data overlay, the data overlay a result of a bioprocessing step associated with the project;
linking the report to the project, the project accessible to at least two users associated with the project; and
tracking changes made by the at least two users to the first data set or the second data set associated with the report.

9. The method of claim 8, wherein the electronic laboratory notebook or the laboratory device receives the report, the report including an integration of a first data file in parallel with a second data file.

10. The method of claim 8, further including linking the first data set to a project to make the first data set searchable in relation to the project.

11. The method of claim 8, further including negotiating at least one parameter between the system for process data management and a data source system.

12. The method of claim 11, wherein the at least one parameter includes an information transfer rate or an interrupt procedure.

13. The method of claim 8, further including modifying the first data set or the second data set data based on a desired assessment or a type of data output, the type of data output a graphical data output or a tabulated data output.

14. The method of claim 8, further including linking the first data set to the second data set based on a user-based search entry, the first data set and the second data set a same data type, the data type including a graphical data type or a tabulated data type.

15. At least one computer readable storage medium comprising instructions that, when executed, cause at least one processor to at least:
retrieve a first data set from at least one of an electronic laboratory notebook or a first laboratory device;
retrieve a second data set from a second laboratory device;
link the first data set and the second data set to a project, the first data and the second data set linked to the project based on a type of data in the first data set and the second data set;
generate a data overlay using the first data set and the second data set, the data overlay including an overlay of a first chromatogram and a second chromatogram;
generate a report including the data overlay, the data overlay a result of a bioprocessing step associated with the project;
link the report to the project, the project accessible to at least two users associated with the project; and
track changes made by the at least two users to the first data set or the second data set associated with the report.

16. The at least one storage medium as defined in claim 15, wherein the computer readable instructions, when executed, cause the one or more processors to negotiate at least one parameter between the system for process data management and a data source system.

17. The at least one storage medium as defined in claim 15, wherein the computer readable instructions, when executed, cause the one or more processors to modify the first data set or the second data set data based on a desired assessment or a type of data output, the type of data output a graphical data output or a tabulated data output.

18. The at least one storage medium as defined in claim 15, wherein the computer readable instructions, when executed, cause the one or more processors to link the first data set to the second data set based on a user-based search entry, the first data set and the second data set a same data type, the data type including a graphical data type or a tabulated data type.

19. The at least one storage medium as defined in claim 15, wherein the electronic laboratory notebook or the laboratory device receives the report, the report including an integration of a first data file in parallel with a second data file.

20. The at least one storage medium as defined in claim 15, wherein the computer readable instructions, when executed, cause the one or more processors link the first data set to a project to make the first data set searchable in relation to the project.
